(19) United States
(12) Patent Application Publication
(10) Pub. No.: US 2023/0400451 A1
(43) Pub. Date: Dec. 14, 2023

(54) METHOD FOR DETECTING IMMUNE EFFICACY AND METHOD OF TREATING A CANCER

(71) Applicant: Chun-Hsuan HO, Taipei City (TW)

(72) Inventor: Chun-Hsuan HO, Taipei City (TW)

(21) Appl. No.: 18/113,237

(22) Filed: Feb. 23, 2023

(30) Foreign Application Priority Data
Jun. 10, 2022 (TW) 111121739
Nov. 2, 2022 (TW) 111141861

(51) Int. Cl. G01N 33/50; G01N 33/569; G16B 40/00; C12N 5/0783; C12N 5/09; A61P 35/00; A61K 35/17

(52) U.S. Cl. CPC G01N 33/5091; G01N 33/56972; G16B 40/00; C12N 5/0637; C12N 5/0693; A61P 35/00; A61K 35/17; C12N 2502/1164; C12N 2502/30

(57) ABSTRACT

In some embodiments of the present disclosure, a method for detecting immune efficacy is provided, including: generating quantity percentages of the plurality of T cell subgroups in the lymphocytes and quantity percentages of the nature killer cell subgroups in the lymphocytes to obtain actual percentages of the plurality of T cell subgroups and actual percentages of the nature killer cell subgroups; and judging whether immune efficacy is normal according to the actual percentages of the plurality of T cell subgroups and the actual percentages of the nature killer cell subgroups. Some embodiments of the present disclosure further provide a method of treating a cancer of an individual by regulating an actual number of a total number of T cells or nature killer cells.

### CROSS-REFERENCE TO RELATED APPLICATION

This application claims priority to Taiwan Application Serial Number 111121739, filed Jun. 10, 2022, and claims priority to Taiwan Application Serial Number 111141861, filed Nov. 2, 2022, both are herein incorporated by reference.

### BACKGROUND

### Field of Invention

The present disclosure relates to a method for detecting immune efficacy and a system therefor, and method of treating a cancer of an individual by lymphocytes or nature killer cells.

### Description of Related Art

Human blood accounts for about one-thirteenth of body weight (that is, there are about 5 L of blood in a human body weighing 65 kg, and lymphocytes account for about 3.5 billion/L to 10 billion/L). Different lymphocytes have different activities and functions, which coordinate with each other to fight against foreign pathogens or internal diseases (e.g., cancer), and maintain physiological balance in the body. However, the current analytical indicators for evaluating immune efficacy are limited, and it is difficult to present a state of the immune system in an all-round way.

Furthermore, the selection of cell amount in immune cell therapy is usually based on the doctor's experience and the types of cells. However, there is a huge gap in the effectiveness of immune cell therapy due to the difference of the immune efficacies of different human individuals, which is also the bottleneck of the current immune cell therapy in clinical treatment.

Therefore, how to provide a more comprehensive method for detecting immune efficacy and how to regulate the immune efficacies of different human individuals to a similar level before immunotherapy are problems to be solved.

### SUMMARY

In some embodiments of the present disclosure, a method for detecting immune efficacy is provided, which includes: providing an in vitro sample of an individual, in which the in vitro sample includes a plurality of lymphocytes, and the plurality of lymphocytes includes T cells and natural killer cells; classifying the T cells into a plurality of T cell subgroups based on expression or not of a plurality of first antigens, and classifying the natural killer cells into a plurality of natural killer cell subgroups based on expression or not of a plurality of second antigens, in which the first antigens include CD28, and the plurality of second antigens include CD16; generating quantity percentages of the plurality of T cell subgroups in the plurality of lymphocytes and quantity percentages of the plurality of nature killer cell subgroups in the plurality of lymphocytes to obtain actual percentages of the plurality of T cell subgroups and actual percentages of the plurality of nature killer cell subgroups; and judging whether the actual percentage of each of the plurality of T cell subgroups meets a T cell subgroup reference range, and judging whether the actual percentage of each of the plurality of natural killer cell subgroups meets a natural killer cell subgroup reference range, when the actual percentage of each of the plurality of T cell subgroups meets the T cell subgroup reference range and the actual percentage of each of the plurality of natural killer cell subgroups meets the natural killer cell subgroup reference range, the immune efficacy of the individual is judged normal.

In some embodiments, the plurality of T cell subgroups include: an naive helper T cell, an aging helper T cell, a regulatory helper T cell, an naive cytotoxic T cell, an aging cytotoxic T cell or a combination thereof, in which the naive helper T cell is CD3+, CD4+, CD45RA+, CD62L+, and CD28+; the aging helper T cell is CD3+, CD4+, CD45RO+, and CD62L+; the regulatory helper T cell is CD3+, CD4+, CD25+, FoXP3+, and CD39+; the naive cytotoxic T cell is CD8+, CD27+, CD45RA+, CD62L+, and CD127+; and the aging cytotoxic T cell is CD8+, CD27+, CD45RO+, CD62L+, and CD57+.

In some embodiments, the T cell subgroup reference range includes a naive helper T cell reference range of from 20% to 100%.

In some embodiments, the T cell subgroup reference range includes a regulatory helper T cell reference range of from 1% to 15%.

In some embodiments, the T cell subgroup reference range includes a naive cytotoxic T cell reference range of from 15% to 100%.

In some embodiments, the T cell subgroup reference range includes an aging cytotoxic T cell reference range of from 0% to 50%.

In some embodiments, the T cell subgroup reference range is derived from physiological values of a healthy population, in which the healthy population is human individuals with ages from 5 to 85 and does not suffer from immunodeficiency syndrome and hyperimmune syndrome.

In some embodiments, the method further includes: generating a quantity percentage of CD28 positive in the naive helper T cell, the aging helper T cell and the regulatory helper T cell relative to a sum of quantity percentages of the naive helper T cell to obtain an actual percentage of CD28 positive in helper T cells, based on a total weight (100% by weight) of the aging helper T cell and the regulatory helper T cell; and judging whether the actual percentage of CD28 positive in the helper T cells meets a CD28 positive reference range in the helper T cells.

In some embodiments, the CD28 positive reference range in the helper T cells is from 80% to 100%.

In some embodiments, the CD28 positive reference range in the helper T cells is derived from physiological values of a healthy population, in which the healthy population is human individuals with ages from 5 to 85 and does not suffer from immunodeficiency syndrome and hyperimmune syndrome.

In some embodiments, the plurality of natural killer cell subgroups include a cytotoxic natural killer cell and a regulatory natural killer cell, in which the cytotoxic natural killer cell is CD16+, CD34+, CD56+, CD94+, and CD117+, and the regulatory natural killer cell is CD34+, CD56+, CD94+, and CD117+.

In some embodiments, the natural killer cell subgroup reference range includes a regulatory natural killer cell reference range of from 0% to 20%.

In some embodiments, the regulatory natural killer cell reference range is derived from physiological values of a healthy population, in which the healthy population is human individuals with ages from 5 to 85 and does not suffer from immunodeficiency syndrome and hyperimmune syndrome.

In some embodiments, the method further includes: determining an actual value of a natural killer cell cytotoxicity of the plurality of natural killer cells, in which the actual value of the natural killer cell cytotoxicity is calculated by following steps: co-culturing the plurality of natural killer cells with a plurality of cancer cells with a quantitative ratio; and generating a quantity percentage of the cancer cells that die to obtain the actual value of the natural killer cell cytotoxicity; and judging whether the actual value of the natural killer cell cytotoxicity meets a natural killer cell cytotoxicity reference range, in which the natural killer cell cytotoxicity reference range is from 0% to 58.8% when the quantitative ratio is 6.25, the natural killer cell cytotoxicity reference range is from 1.7% to 88.7% when the quantitative ratio is 12.5, the natural killer cell cytotoxicity reference range is from 17.4% to 100% when the quantitative ratio is 25, and the natural killer cell cytotoxicity reference range is from 35.3% to 100% when the quantitative ratio is 50.

In some embodiments, the method further includes: cryopreserving each of the plurality of T cell subgroups when the actual percentage of each of the plurality of T cell subgroups meets the T cell subgroup reference range; and cryopreserving each of the plurality of natural killer cell subgroups when the actual percentage of each of the plurality of natural killer cell subgroups meets the natural killer cell subgroup reference range.

In some embodiments, cryopreserving each of the plurality of T cell subgroups or cryopreserving each of the plurality of natural killer cell subgroups includes: sorting each of the plurality of T cell subgroups or each of the plurality of natural killer cell subgroups by an immunomagnetic bead cell sorting method; and cryopreserving each of the plurality of T cell subgroups or each of the plurality of natural killer cell subgroups.

In some embodiments of the present disclosure, a system for detecting immune efficacy is provided, which includes a processor and a memory, and the memory stores a plurality of computer program instructions, and the computer program instructions, when executed by the processor, cause the processor to implement following steps: accessing an in vitro sample data of an individual, the in vitro sample data including a plurality of sample cell surface antigen data; generating a plurality of T cell subgroup count data and a plurality of natural killer cell subgroup count data according to the in vitro sample data using a cell subgroup database, in which the cell subgroup database includes a T cell subgroup classification information and a natural killer cell subgroup classification information, and the T cell subgroup classification information includes a classification index according to expression or not of a plurality of first antigens, in which the plurality of first antigens include CD28, and the natural killer cell subgroup classification information includes a classification index according to expression or not of a plurality of second antigens, in which the plurality of second antigens include CD16; and generating an immune efficacy data according to the plurality of T cell subgroup count data and the plurality of natural killer cell subgroup count data using an immune efficacy evaluation database, in which the immune efficacy evaluation database includes a plurality of T cell subgroup reference range data and a plurality of natural killer cell subgroup reference range data.

In some embodiments, the plurality of first antigens further include CD3, CD4, CD8, CD25, CD27, CD39, CD45RO, CD45RA, CD57, CD62L, CD127, FoxP3, or a combination thereof, and the plurality of second antigens further include CD34, CD56, CD94, CD117, or a combination thereof.

In some embodiments, the system further includes an output module connected to the processor, and the output module receiving the immune efficacy data and outputs an immune efficacy report.

In some embodiments, the immune efficacy report includes an immune efficacy field and a plurality of immune analysis index fields.

In some embodiments of the present disclosure, a method of treating a cancer of an individual by a plurality of lymphocytes is provided, in which the plurality of lymphocytes include a plurality of T cells, a plurality of natural killer cells or a combination thereof, and the method includes: (a) generating a deviation value of a total number of T cells between an actual number of the total number of T cells and a theoretical range of the total number of T cells, and generating a deviation value of a total number of nature killer cells between an actual number of the total number of nature killer cells and a theoretical range of the total number of nature killer cells; and (b) administering the pharmaceutical composition including the plurality of T cells to the individual based on the deviation value of the total number of T cells to allow the actual number of the total number of T cells of the individual to fall within the theoretical range of the total number of T cells, and administering the pharmaceutical composition including the plurality of nature killer cells to the individual based on the deviation value of the total number of nature killer cells to allow the actual number of the total number of nature killer cells of the individual to fall within the theoretical range of the total number of nature killer cells.

In some embodiments, the theoretical range of the total number of T cells and the theoretical range of the total number of nature killer cells are derived from physiological values of a healthy population, in which the healthy population is human individuals with ages from 5 to 85 and does not suffer from immunodeficiency syndrome and hyperimmune syndrome.

In some embodiments, the theoretical range of the total number of T cells is from 700 cells/μL to 2500 cells/μL, and the theoretical range of the total number of nature killer cells is from 100 cells/μL to 300 cells/μL.

In some embodiments, the method further includes: (a) classifying the plurality of lymphocytes into a plurality of T cell subgroups based on expression or not of a plurality of first antigens, and classifying the plurality of lymphocytes into a plurality of natural killer cell subgroups based on expression or not of a plurality of second antigens; (b) generating a deviation value of a T cell subgroup between an actual percentage of the T cell subgroup and a theoretical range of the T cell subgroup, and generating a deviation value of a nature killer cell subgroup between an actual percentage of the nature killer cell subgroup and a theoretical range of the nature killer cell subgroup, in which the actual percentage of the T cell subgroup is an actual quantity percentage of each of the plurality of T cell subgroups in the plurality of lymphocytes, and the actual percentage of the nature killer cell subgroup is an actual quantity percentage of each of the plurality of nature killer cell subgroups in the plurality of lymphocytes; and (c) administering the pharmaceutical composition including the plurality of T cell subgroups to the individual based on the deviation value of the T cell subgroup to allow the actual percentage of the T cell subgroup of the individual to fall within the theoretical range of the T cell subgroup, and administering the pharmaceutical composition including the plurality of nature killer cells to the individual based on the deviation value of the nature killer cell subgroup to allow the actual percentage of the nature killer cell subgroup of the individual to fall within the theoretical range of the nature killer cell subgroup.

In some embodiments, the plurality of T cell subgroups include: a naive helper T cell, a regulatory helper T cell, an naive cytotoxic T cell, or a combination thereof, in which the naive helper T cell is CD3+, CD4+, CD45RA+, CD62L+, and CD28+; the regulatory helper T cell is CD3+, CD4+, CD25+, FoxP3+, and CD39+; and the naive cytotoxic T cell is CD8+, CD27+, CD45RA+, CD62L+, and CD127+.

In some embodiments, the theoretical range of the T cell subgroup includes a theoretical range of the naive helper T cell, a theoretical range of the regulatory helper T cell, a theoretical range of the naive cytotoxic T cell, or a combination thereof, in which the theoretical range of the naive helper T cell is from 35% to 100%, the theoretical range of the regulatory helper T cell is from 3% to 10%, and the theoretical range of the naive cytotoxic T cell is from 20% to 100%.

In some embodiments, the plurality of natural killer cell subgroups include a regulatory natural killer cell, in which the regulatory natural killer cell is CD34+, CD56+, CD94+, and CD117+.

In some embodiments, the theoretical range of the nature killer cell subgroup includes a theoretical range of the regulatory natural killer cell, in which the theoretical range of the regulatory natural killer cell is from 0% to 10%.

In some embodiments, the method further includes: (a) classifying the plurality of lymphocytes into a plurality of T cell subgroups based on expression or not of a plurality of first antigens, in which the plurality of T cell subgroups include a first T cell subgroup and a second T cell subgroup; (b) generating a deviation value of a ratio of T cell subgroups between an actual value of a ratio of two kinds of T cell subgroups and a theoretical range of the ratio of two kinds of T cell subgroups, in which the actual value of the ratio of two kinds of T cell subgroups is a quantitative ratio of the first T cell subgroup and the second T cell subgroup; and (c) administering the pharmaceutical composition including the first T cell subgroup or the second T cell subgroup to the individual based on the deviation value of the ratio of the T cell subgroups to allow the actual value of the ratio of two kinds of T cell subgroups of the individual to fall within the theoretical range of the ratio of two kinds of T cell subgroups.

In some embodiments, the theoretical range of the ratio of two kinds of T cell subgroups is derived from physiological values of a healthy population, in which the healthy population is human individuals with ages from 5 to 85 and does not suffer from immunodeficiency syndrome and hyperimmune syndrome.

In some embodiments, the theoretical range of the ratio of two kinds of T cell subgroups is from 1:1 to 5:1 when the first T cell subgroup is CD4 positive T cells and the second T cell subgroup is CD8 positive T cells.

In some embodiments, the method further includes performing an immune cell therapy on the individual after the step of administering the pharmaceutical composition including the plurality of T cell subgroups to the individual or administering the pharmaceutical composition including the plurality of nature killer cells to the individual.

In some embodiments, the immune cell therapy includes a natural killer cell therapy, a cytokine-induced killer cell therapy, a γδ T cell therapy, a dendritic cell therapy, a tumor infiltrating lymphocyte therapy, a chimeric antigen receptor T cell therapy, or a combination thereof.

In some embodiments, the plurality of lymphocytes are derived from the individual and obtained by culturing in vitro.

In some embodiments, the cancer includes colorectal cancer, ovarian cancer, prostate cancer, breast cancer, brain cancer, cervical cancer, bladder cancer, anal cancer, uterine cancer, colon cancer, liver cancer, pancreatic cancer, lung cancer, endometrial cancer, bone cancer, testicular cancer, skin cancer, kidney cancer, stomach cancer, esophagus cancer, head and neck cancer, salivary gland cancer, hepatocellular carcinoma, non-small cell lung cancer, head and neck squamous cell cancer, basal cell cancer, cutaneous squamous cell cancer, cholangiocarcinoma, merkel cell carcinoma or a combination thereof.

In some embodiments of the present disclosure, a method of treating a cancer of an individual by a plurality of nature killer cells is provided, in which the method includes: (a) determining an actual value of a natural killer cell cytotoxicity of the plurality of natural killer cells, in which the actual value of the natural killer cell cytotoxicity is generated by following steps: co-culturing the plurality of natural killer cells with a plurality of cancer cells with a quantitative ratio; and generating a quantity percentage of the cancer cells that die to obtain the actual value of the natural killer cell cytotoxicity; (b) generating a deviation value of a natural killer cell cytotoxicity between an actual value of the natural killer cell cytotoxicity and a theoretical range of the natural killer cell cytotoxicity based on the actual value of the natural killer cell cytotoxicity and the theoretical range of the natural killer cell cytotoxicity; (c) administering the pharmaceutical composition including the plurality of nature killer cells to the individual based on the deviation value of the natural killer cell cytotoxicity to allow the actual value of the natural killer cell cytotoxicity of the individual to fall within the theoretical range of the natural killer cell cytotoxicity.

In some embodiments, the theoretical range of the natural killer cell cytotoxicity is from 0% to 58.8% when the quantitative ratio is 6.25, the theoretical range of the natural killer cell cytotoxicity is from 1.7% to 88.7% when the quantitative ratio is 12.5, the theoretical range of the natural killer cell cytotoxicity is from 17.4% to 100% when the quantitative ratio is 25, and the theoretical range of the natural killer cell cytotoxicity is from 35.3% to 100% when the quantitative ratio is 50.

In some embodiments, at the step of (a), the theoretical range of the natural killer cell cytotoxicity is 57.4% to 100% when the quantitative ratio is 50, and interleukin-2 with an action concentration of 100 international units/mL is added to co-culture with the plurality of natural killer cells and the cancer cells.

In some embodiments, the method further includes: generating a deviation value of a total number of nature killer cells between an actual number of the total number of nature killer cells and a theoretical range of the total number of nature killer cells; and administering the pharmaceutical composition including the plurality of nature killer cells to the individual based on the deviation value of the natural killer cell cytotoxicity and the deviation value of the total number of nature killer cells to allow the actual number of the total number of nature killer cells of the individual to fall within the theoretical range of the total number of nature killer cells, thereby allowing the actual value of the natural killer cell cytotoxicity to fall within the theoretical range of the natural killer cell cytotoxicity.

In some embodiments, the method further includes: classifying the plurality of nature killer cells into a plurality of natural killer cell subgroups based on expression or not of a plurality of antigens, wherein the plurality of natural killer cell subgroups include a regulatory natural killer cell, wherein the regulatory natural killer cell is CD34+, CD56+, CD94+, and CD117+; generating a deviation value of a regulatory natural killer cell between an actual percentage of the regulatory natural killer cell and a theoretical range of the regulatory natural killer cell, wherein the actual percentage of the regulatory natural killer cell is an actual quantity percentage of the regulatory natural killer cell in lymphocytes; administering the pharmaceutical composition including the regulatory natural killer cell to the individual based on the deviation value of the natural killer cell cytotoxicity and the deviation value of the regulatory natural killer cell to allow the actual percentage of the regulatory natural killer cell of the individual to fall within the theoretical range of the regulatory natural killer cell, thereby allowing the actual value of the natural killer cell cytotoxicity to fall within the theoretical range of the natural killer cell cytotoxicity.

### BRIEF DESCRIPTION OF THE DRAWINGS

In order to make the above-mentioned and other objects, features, advantages and embodiments of the present disclosure more clearly understood, descriptions of accompanying drawings are as follows:

FIG. 1 illustrates a flow chart of a method for detecting immune efficacy in some embodiments of the present disclosure.

FIG. 2 illustrates a flow chart of steps implemented by a processor in a system for detecting immune efficacy in some embodiments of the present disclosure.

FIG. 3 illustrates a schematic diagram of a system for detecting immune efficacy in some embodiments of the present disclosure.

### DETAILED DESCRIPTION

In order that the present disclosure is described in detail and completeness, implementation aspects and specific embodiments of the present disclosure with illustrative description are presented, but those are not the only form for implementation or use of the specific embodiments of the present disclosure. The embodiments disclosed herein may be combined or substituted with each other in an advantageous manner, and other embodiments may be added to an embodiment without further description. In the following description, numerous specific details will be described in detail in order to enable the reader to fully understand the following embodiments. However, the embodiments of the present disclosure may be practiced without these specific details.

In this description, unless the context specifically dictates otherwise, “a” and “the” may mean a single or a plurality. It will be further understood that “comprise”, “include”, “have”, and similar terms as used herein indicate described features, regions, integers, steps, operations, elements and/or components, but not exclude other features, regions, integers, steps, operations, elements, components and/or groups.

In this description, a deviation value represents a difference obtained by subtracting two numbers. For example, a deviation value of a total number of T cells between an actual number of a total number of T cells and a theoretical range of a total number of T cells means the difference obtained by subtracting the theoretical range of the total number of T cells from the actual number of the total number of T cells.

Although a series of operations or steps are described below to illustrate the method disclosed herein, the order of the operations or steps is not to be construed as limiting. For example, certain operations or steps may be performed in a different order and/or concurrently with other steps. In addition, not all illustrated operations, steps, and/or features are required to implement embodiments of the present disclosure. Moreover, each of the operations or steps described herein may include a plurality of sub-steps or actions.

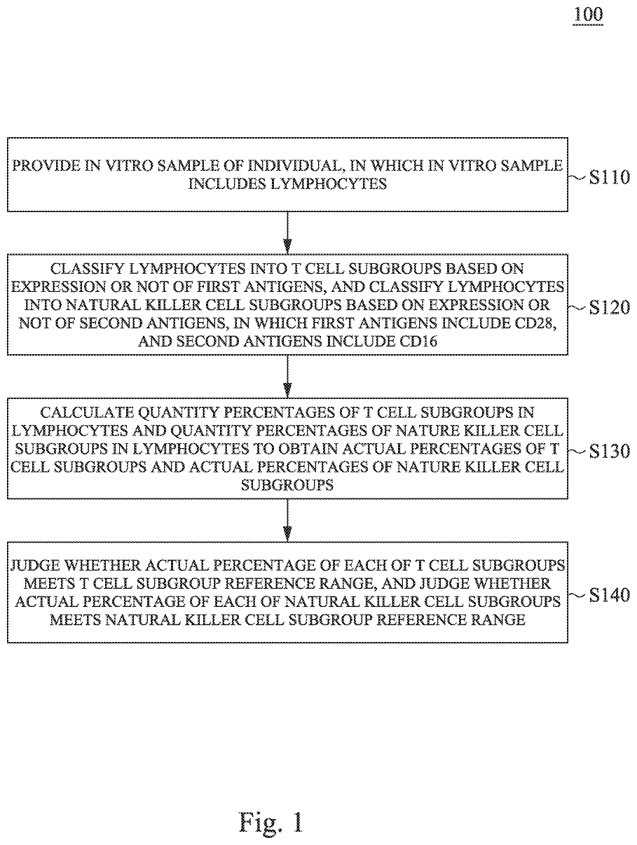

Please refer to FIG. 1, some embodiments of the present disclosure provide a method 100 for detecting immune efficacy, which includes steps S110 to S140.

First, in step S110, an in vitro sample of an individual is provided, in which the in vitro sample includes a plurality of lymphocytes, in which the lymphocytes include T cells and natural killer cells.

In some embodiments, the in vitro sample is human blood.

Next, in step S120, the lymphocytes are classified into a plurality of T cell subgroups according to expression or not of a plurality of first antigens, and the lymphocytes are classified into a plurality of natural killer cell subgroups according to expression or not of a plurality of second antigens, in which the first antigens include CD28 and the second antigens include CD16.

In some embodiments, the first antigens further include CD3, CD4, CD8, CD25, CD27, CD39, CD45RO, CD45RA, CD57, CD62L, CD127, FoxP3, or a combination thereof. In some embodiments, the second antigens further include CD34, CD56, CD94, CD117, or a combination thereof.

It should be noted that CD28 is a surface antigen of an initial helper cell in the T cell subgroups, and CD16 is a surface antigen of a cytotoxic natural killer cell. Therefore, according to step S120, the T cell subgroups can be classified into naive and non-naive helper cells, and the natural killer cell subgroups can be classified into cytotoxic and non-cytotoxic natural killer cells.

In some embodiments, classifying the T cells into the T cell subgroups includes: classifying the T cells into naive helper T cells (CD3+CD4+CD45RA+CD62L+CD28+), aging helper T cells (CD3+CD4+CD45RO+CD62L+), regulatory helper T cells (CD3+CD4+CD25+FoxP3+CD39+), naive cytotoxic T cells (CD8+CD27+CD45RA+CD62L+CD127+), aging cytotoxic T cells (CD8+CD27+CD45RO+CD62L+CD57+) or a combination thereof.

In some embodiments, the natural killer cell subgroups include cytotoxic natural killer cells (CD16+CD34+CD56+CD94+CD117+) and regulatory natural killer cells (CD34+CD56+CD94+CD117+).

Next, step S130, quantity percentages of the T cell subgroups in the lymphocytes and quantity percentages of the nature killer cell subgroups in the lymphocytes are respectively calculated to obtain actual percentages of the T cell subgroups and actual percentages of the nature killer cell subgroups. In some embodiments, the actual percentages of the T cell subgroups include an actual percentage of the naive helper T cells, an actual percentage of the aging helper T cells, an actual percentage of the regulatory helper T cells, an actual percentage of the naive cytotoxic T cells, an actual percentage of the aging cytotoxic T cells or a combination thereof. In some embodiments, the actual percentages of the natural killer cell subgroups include an actual percentage of the cytotoxic natural killer cells, an actual percentage of the regulatory natural killer cells, or a combination thereof.

Next, in step S140, it is judged whether the actual percentages of the T cell subgroups meet T cell subgroup reference ranges, respectively, and whether the actual percentages of the natural killer cell subgroups meet natural killer cell subgroup reference ranges, respectively.

In some embodiments, the T cell subgroup reference ranges and the natural killer cell subgroup reference ranges are derived from physiological values of a healthy population, in which the healthy population is human individuals with ages from 5 to 85 and does not suffer from immunodeficiency syndrome (e.g., acquired immunodeficiency syndrome) and hyperimmune syndrome (e.g., autoimmune disease).

In some embodiments, the T cell subgroup reference range or the natural killer cell subgroup reference range includes a corresponding tolerance range and a theoretical range, i.e., the T cell subgroup reference range includes a T cell subgroup tolerance range and a T cell subgroup theoretical range, and the natural killer cell subgroup reference range includes a natural killer cell subgroup tolerance range and a natural killer cell subgroup theoretical range.

When the actual percentages of all of the T cell subgroups respectively meet the corresponding T cell subgroup reference ranges, and the actual percentages of all of the natural killer cell subgroups respectively meet the corresponding natural killer cell subgroup reference ranges (e.g., the actual percentages of all of the T cell subgroups and the actual percentages of all of the natural killer cell subgroups meet the tolerance ranges or theoretical ranges, respectively), it is judged that immune efficacy is normal. In some embodiments, when the actual percentages of all of the T cell subgroups meet the corresponding T cell subgroup theoretical ranges, and the actual percentages of all of the natural killer cell subgroups meet the corresponding natural killer cell subgroup theoretical ranges, it is further judged that immune efficacy is good.

When the actual percentage of any T cell subgroup does not meet the corresponding T cell subgroup reference range, or the actual percentage of the natural killer cell subgroup does not meet the natural killer cell subgroup reference range, it is judged that immune efficacy is poor.

In some embodiments, the T cell subgroup reference range includes a naive helper T cell reference range, a regulatory helper T cell reference range, a naive cytotoxic T cell reference range, or an aging cytotoxic T cell reference range. The naive helper T cell reference range is from 20% to 100%, in which 20%≤ a tolerance range<35%, and a theoretical range is from 35% to 100%. The regulatory helper T cell reference range is from 1% to 15%, in which 1%≤ a tolerance range<3%, and 10%< a tolerance range≤15%, and a theoretical range is from 3% to 10%. The naive cytotoxic T cell reference range is from 15% to 100%, in which 15%≤ a tolerance range<20%, and a theoretical range is from 20% to 100%. The aging cytotoxic T cell reference range is from 0% to 50%, in which 30%< a tolerance range≤50%, and a theoretical range is from 0% to 30%.

In some embodiments, the natural killer cell subgroup reference range includes a regulatory natural killer cell reference range. The regulatory natural killer cell reference range is from 0% to 20%, in which 10%< a tolerance range≤20%, and a theoretical range is from 0% to 10%.

In addition, it is possible to analyze the immune system's defense against diseases or the risk of developing a specific disease based on whether the actual percentages of the T cell subgroups and the actual percentages of the natural killer cell subgroups meet the corresponding T cell subgroup reference ranges and the natural killer cell subgroup reference ranges, respectively.

For example, the actual percentages of the naive helper T cell and the naive cytotoxic T cell fall within the reference ranges (the naive helper T cell reference range or the naive cytotoxic T cell reference range), indicating that it has good protection against novel pathogens. The actual percentage of the naive cytotoxic T cell falls within the naive cytotoxic T cell reference range, indicating that it has better inhibitory ability against cancer. If the actual percentage of the regulatory helper T cell is higher than the regulatory helper T cell reference range, it will over-inhibit other immune cells, resulting in higher mortality in cancer. If the actual percentage of the aging cytotoxic T cell is higher than the aging cytotoxic T cell reference range, indicating that the immune system is aging or declining. If the actual percentage of the regulatory natural killer cell is higher than the regulatory natural killer cell reference range, it will over-inhibit activity of the cytotoxic natural killer cell, and individuals are more likely to be infected with chronic viruses.

In some embodiments, the method 100 further includes generating a quantity percentage of CD28 positive in the naive helper T cell, the aging helper T cell and the regulatory helper T cell relative to a sum of quantity percentages of the naive helper T cell, the aging helper T cell and the regulatory helper T cell taken as 100% (a proportion of CD28 positive (CD28+) in the helper T cells, that is, a quantity percentage of the naive helper T cell in the helper T cells) to obtain an actual percentage of CD28 positive in the helper T cells; and judging whether the actual percentage of CD28 positive in the helper T cells meets a CD28 positive reference range in the helper T cells, so as to determine whether an individual is at risk of developing autoimmune diseases.

In some embodiments, the CD28 positive reference range in the helper T cells is derived from physiological values of a healthy population, in which the healthy population is human individuals with ages from 5 to 85 and does not suffer from immunodeficiency syndrome and hyperimmune syndrome.

In some embodiments, the CD28 positive (CD28+) reference range in the helper T cells is greater than from 80% to 100%, in which 80%≤ a tolerance range<90%, and a theoretical range is from 90% to 100%. Therefore, when the actual percentage of CD28 positive (CD28+) in the helper T cells is less than 80%, the risk of developing autoimmune diseases is high.

In some embodiments, the method 100 further includes determining an actual value of natural killer cell cytotoxicity of the natural killer cells (the actual value of the natural killer cell cytotoxicity is calculated by following steps: co-culturing the natural killer cells with a plurality of cancer cells with a quantitative ratio; and generating a quantity percentage of the cancer cells that die to obtain the actual value of the natural killer cell cytotoxicity), and judging whether the actual value of the natural killer cell cytotoxicity meets a natural killer cell cytotoxicity reference range, in which when the actual value of the natural killer cell cytotoxicity falls within the natural killer cell cytotoxicity reference range, indicating that it has better inhibitory ability to cancer. For example, the natural killer cell cytotoxicity reference range is from 0% to 58.8% when the quantitative ratio is 6.25; the natural killer cell cytotoxicity reference range is from 1.7% to 88.7% when the quantitative ratio is 12.5; the natural killer cell cytotoxicity reference range is from 17.4% to 100% when the quantitative ratio is 25; the natural killer cell cytotoxicity reference range is from 35.3% to 100% when the quantitative ratio is 50. In some embodiments, the natural killer cell cytotoxicity reference range is derived from physiological values of a healthy population, in which the healthy population is human individuals with ages from 5 to 85 and does not suffer from immunodeficiency syndrome and hyperimmune syndrome.

In some embodiments, the natural killer cells and human immortalized myeloid leukemia cells (K-562 cells) with different quantitative ratios are co-cultured, and a proportion of the K-562 cells that die is determined to evaluate the natural killer cell cytotoxicity, in which the quantitative ratios of IL-2 and K-562 cells may be 6.25, 12.5, 25 or 50.

In addition, activity of the regulatory natural killer cells may be completely inhibited by adding interleukin 2 (IL-2) (the regulatory natural killer cells will inhibit activity of the cytotoxic natural killer cells) to obtain activity of the cytotoxic natural killer cells to further analyze maximum activity of the natural killer cells.

In some embodiments, the method 100 further includes generating a content of the T cells and a content of the natural killer cells in the in vitro sample (e.g., how many T cells and natural killer cells are present per milliliter of blood) to analyze an immune system profile. For example, when a total number of the natural killer cells (the number of the natural killer cells per microliter of a whole blood sample) is less than 50 (excluding 50), the risk of developing cancer is high.

In some embodiments, the method 100 further includes cryopreserving the T cell subgroups when the actual percentages of the T cell subgroups meet the T cell subgroup reference ranges, respectively; and cryopreserving the natural killer cell subgroups when the actual percentages of the natural killer cell subgroups meet the natural killer cell subgroup reference ranges, respectively.

In some embodiments, when the actual percentages of the natural killer cell subgroups and the actual value of the natural killer cell cytotoxicity fall within the corresponding reference ranges, the natural killer cell subgroups (e.g., the cytotoxic natural killer cells and the regulatory natural killer cells) are cryopreserved.

In some embodiments, the T cell subgroups or the natural killer cell subgroups (e.g., the T cell subgroups such as the naive helper T cells, the aging helper T cells, the regulatory helper T cells, the naive cytotoxic T cells, or the aging cytotoxic T cells, or the natural killer cell subgroups such as the cytotoxic natural killer cells or the regulatory natural killer cells) may be sorted by an immunomagnetic bead cell sorting method; and the T cell subgroups or the natural killer cell subgroups are cryopreserved. It is worth emphasizing that conventional methods such as immunoprecipitation or centrifugal phase method easily damage cells and make it impossible to continue subculture or cryopreservation. Relatively speaking, the cells isolated by the immunomagnetic bead cell sorting method still have activity after multiple sorting, which is conducive to expansion and preservation.

In some embodiments, a positive sorting system, a negative sorting system, or a combination thereof can be used to sort the T cell subgroups or the natural killer cell subgroups. In some embodiments, CD3, CD4, CD8, CD25, CD27, CD28, CD39, CD45, CD45RO, CD45RA, CD57, CD62L, CD127, FoxP3, or a combination thereof can be used to sort out the naive helper T cells, the aging helper T cells, the regulatory helper T cells, the naive cytotoxic T cells, and the aging cytotoxic T cells. In some embodiments, CD16, CD34, CD56, CD94, CD117, or a combination thereof can be used to sort out the cytotoxic natural killer cells and the regulatory natural killer cells.

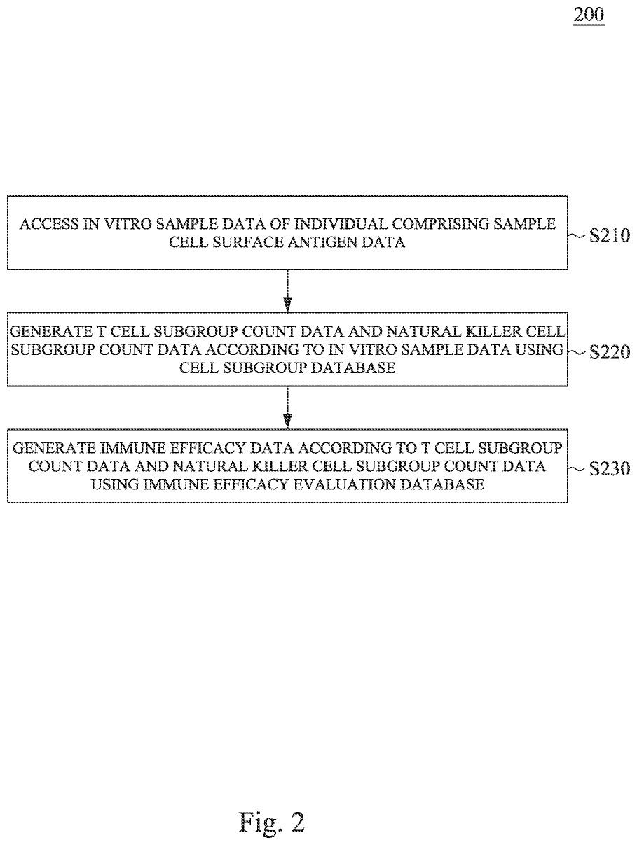

Some embodiments of the present disclosure also provide a system for detecting immune efficacy, which includes a processor and a memory. The memory stores a plurality of computer program instructions. The computer program instructions, when executed by the processor, cause the processor to implement a process 200 including step S210 to step S230, please refer to FIG. 2.

First, in step S210, an in vitro sample data of an individual is accessed, and the in vitro sample data includes a plurality of sample cell surface antigen data. In some embodiments, the sample cell surface antigen data is analyzed by flow cytometer.

Next, please refer to step S220, a plurality of T cell subgroup count data and a plurality of natural killer cell subgroup count data are generated according to the in vitro sample data using a cell subgroup database. The cell subgroup database includes a T cell subgroup classification information and a natural killer cell subgroup classification information, and the T cell subgroup classification information includes a classification index according to expression or not of a plurality of first antigens, in which the first antigens include CD28, and the natural killer cell subgroup classification information includes a classification index according to expression or not of a plurality of second antigens, in which the second antigens include CD16.

In some embodiments, the first antigens further include CD3, CD4, CD8, CD25, CD27, CD39, CD45RO, CD45RA, CD57, CD62L, CD127, FoxP3, or a combination thereof, and the second antigens further include CD34, CD56, CD94, CD117, or a combination thereof.

In some embodiments, the T cell subgroup classification information includes antigen expression information of T cell subgroups, for example, the naive helper T cells are CD3+CD4+CD45RA+CD62L+CD28+. The natural killer cell subgroup classification information includes antigen expression information of natural killer cell subgroups, for example, the regulatory natural killer cells are CD34+CD56+CD94+CD117+.

In some embodiments, the T cell subgroup count data and the natural killer cell subgroup count data include quantity percentages of the T cell subgroups or the natural killer cell subgroups in lymphocytes, such as a quantity percentage of the naive helper T cells in the lymphocytes.

Next, please refer to step S230, an immune efficacy data is generated according to the T cell subgroup count data and the natural killer cell subgroup count data using an immune efficacy evaluation database. The immune efficacy evaluation database includes a plurality of T cell subgroup reference range data (e.g., including the T cell subgroup reference ranges shown in the aforementioned method 100) and a plurality of natural killer cell subgroup reference range data (e.g., including the natural killer cell subgroup reference ranges shown in the aforementioned method 100).

In some embodiments, the system further includes an output module connected to the processor, and the output module receives the immune efficacy data and outputs an immune efficacy report (e.g., displayed on a display screen) for evaluating immune efficacy of the individual, and determines whether to cryopreserve the cells, in which the immune efficacy report includes an immune efficacy field (recording a level of the immunity efficacy) and a plurality of immune analysis index fields (including proportions of the cell subgroups).

In some embodiments, the T cell subgroup reference range data or the natural killer cell subgroup reference range data includes a corresponding tolerance range data and a theoretical range data, that is, the T cell subgroup reference range data includes a T cell subgroup tolerance range data and a T cell subgroup theoretical range data, and the natural killer cell subgroup reference range data includes a natural killer cell subgroup tolerance range data and a natural killer cell subgroup theoretical range data.

When all of the T cell subgroup count data meet the corresponding T cell subgroup reference range data, and all of the natural killer cell subgroup count data meet the corresponding natural killer cell subgroup reference range data (e.g., all of the T cell subgroup count data and all of the natural killer cell subgroup count data meet the tolerance range data or the theoretical range data, respectively), immune efficacy is recorded as normal in the immune efficacy field. In some embodiments, when all of the T cell subgroup count data meet the corresponding T cell subgroup theoretical range data, and all of the natural killer cell subgroup count data meet the corresponding natural killer cell subgroup theoretical range count data, it is further judged that immune efficacy is good.

When any of the T cell subgroup count data does not meet the corresponding T cell subgroup reference range data, or the natural killer cell subgroup count data does not meet the natural killer cell subgroup reference range data, it is judged that immune efficacy is poor.

In some other embodiments, T cell subgroup reference range data or the natural killer cell subgroup count data is respectively the theoretical range count data.

In some embodiments of the present disclosure, a method of treating a cancer of an individual by a plurality of lymphocytes, in which the plurality of lymphocytes include a plurality of T cells, a plurality of natural killer cells or a combination thereof, and the method includes: (a) generating a deviation value of a total number of T cells between an actual number of the total number of T cells and a theoretical range of the total number of T cells, and generating a deviation value of a total number of nature killer cells between an actual number of the total number of nature killer cells and a theoretical range of the total number of nature killer cells; and (b) administering the pharmaceutical composition including the plurality of T cells to the individual based on the deviation value of the total number of T cells to allow the actual number of the total number of T cells of the individual to fall within the theoretical range of the total number of T cells, and administering the pharmaceutical composition including the plurality of nature killer cells to the individual based on the deviation value of the total number of nature killer cells to allow the actual number of the total number of nature killer cells of the individual to fall within the theoretical range of the total number of nature killer cells.

It is understood that the individual suffering from a cancer often has insufficient immunity due to insufficient actual number of the total number of T cells, insufficient actual number of the total number of natural killer cells, or both. The present disclosure regulates the performance of the individual's immune system to a healthy state by adjusting the actual number of the total number of T cells and the actual number of the total number of nature killer cells to the theoretical range, and the individual's immune system against the cancer is increased, thereby improving the therapeutic effect of the cancer. Compared with traditional radiation therapy or chemotherapy, the use of the present disclosure will not cause damage to the individual's cells. In addition, the use of this disclosure can also be used in combination with other immune cell therapies, the effectiveness of immune cell therapy is increased through precise regulation of the actual number of the total number of T cells and the actual number of the total number of natural killer cells, and the side effects such as the overreaction (for example, cytokine storm) caused by miscalculation of the actual number of the total number of T cells or the actual number of the total number of natural killer cells are avoided.

In some embodiments, the theoretical range of the total number of T cells and the theoretical range of the total number of nature killer cells are derived from physiological values of a healthy population, in which the healthy population is human individuals with ages from 5 to 85 and does not suffer from immunodeficiency syndrome and hyperimmune syndrome.

In some embodiments, the theoretical range of the total number of T cells is from 700 cells/μL to 2500 cells/μL (such as 700 cells/μL, 800 cells/μL, 900 cells/μL, 1000 cells/μL, 1100 cells/μL, 1200 cells/μL, 1300 cells/μL, 1400 cells/μL, 1500 cells/μL, 1600 cells/μL, 1700 cells/μL, 1800 cells/μL, 1900 cells/μL, 2000 cells/μL, 2100 cells/μL, 2200 cells/μL, 2300 cells/μL, 2400 cells/μL, 2500 cells/μL or a value between the aforementioned intervals), and the theoretical range of the total number of nature killer cells is from 100 cells/μL to 300 cells/μL (such as 100 cells/μL, 200 cells/μL, 300 cells/μL or a value between the aforementioned intervals). If the actual number of the total number of T cells is less than the theoretical range of the total number of T cells, the actual number of the total number of nature killer cells is less than the theoretical range of the total number of nature killer cells, or both conditions exist simultaneously, the immune efficacy of the individual is low. Conversely, if the actual number of the total number of T cells is greater than the theoretical range of the total number of T cells, the actual number of the total number of nature killer cells is greater than the theoretical range of the total number of natural killer cells, or both conditions exist simultaneously, a state of excessive autoimmunity may exist.

In some embodiments, the appropriate dose of T cells or natural killer cells should be administered according to the individual's blood content (for example, a 65 kg individual with the blood volume of about 5 L) in combination with the deviation of the total number of T cells or the deviation of the total number of natural killer cells.

In some embodiments, the method further includes: (a) classifying the plurality of lymphocytes into a plurality of T cell subgroups based on expression or not of a plurality of first antigens, and classifying the plurality of lymphocytes into a plurality of natural killer cell subgroups based on expression or not of a plurality of second antigens; (b) generating a deviation value of a T cell subgroup between an actual percentage of the T cell subgroup and a theoretical range of the T cell subgroup, and generating a deviation value of a nature killer cell subgroup between an actual percentage of the nature killer cell subgroup and a theoretical range of the nature killer cell subgroup, in which the actual percentage of the T cell subgroup is an actual quantity percentage of each of the plurality of T cell subgroups in the plurality of lymphocytes, and the actual percentage of the nature killer cell subgroup is an actual quantity percentage of each of the plurality of nature killer cell subgroups in the plurality of lymphocytes; and (c) administering the pharmaceutical composition including the plurality of T cell subgroups to the individual based on the deviation value of the T cell subgroup to allow the actual percentage of the T cell subgroup of the individual to fall within the theoretical range of the T cell subgroup, and administering the pharmaceutical composition including the plurality of nature killer cells to the individual based on the deviation value of the nature killer cell subgroup to allow the actual percentage of the nature killer cell subgroup of the individual to fall within the theoretical range of the nature killer cell subgroup.

It is worth emphasizing that, compared with adjusting the actual number of the total number of T cells or the actual number of the total number of nature killer cells, administering a pharmaceutical composition including the T cell subgroup or the natural killer cell subgroup to the individual according to the deviation value of the T cell subgroup or the deviation value of the natural killer cell subgroup realizes the more precise regulation of immune efficacy by further adjusting the T cell subgroup and the natural killer cell subgroup which mainly control anti-cancer cytotoxicity in the immune system.

In some embodiments, the theoretical range of the T cell subgroup and the theoretical range of the nature killer cell subgroup are derived from physiological values of a healthy population, in which the healthy population is human individuals with ages from 5 to 85 and does not suffer from immunodeficiency syndrome and hyperimmune syndrome.

In some embodiments, the appropriate dose of the T cell subgroup or natural killer cell subgroup should be administered according to the individual's blood content (for example, a 65 kg individual with the blood volume of about 5 L) in combination with the deviation of the T cell subgroup and the actual number of the total number of T cells or the deviation of the nature killer cell subgroup and the actual number of the total number of nature killer cells.

In some embodiments, the plurality of first antigens further include CD3, CD4, CD8, CD25, CD27, CD39, CD45RO, CD45RA, CD57, CD62L, CD127, FoxP3, or a combination thereof. In some embodiments, the plurality of second antigens include CD16, CD34, CD56, CD94, CD117, or a combination thereof. In some embodiments, classifying the T cells into the T cell subgroups includes: classifying the T cells into naive helper T cells (CD3+CD4+CD45RA+CD62L+CD28+), aging helper T cells (CD3+CD4+CD45RO+CD62L+), regulatory helper T cells (CD3+CD4+CD25+FoxP3+CD39+), naive cytotoxic T cells (CD8+CD27+CD45RA+CD62L+CD127+), aging cytotoxic T cells (CD8+CD27+CD45RO+CD62L+CD57+) or a combination thereof.

In some embodiments, the natural killer cell subgroups include cytotoxic natural killer cells (CD16+CD34+CD56+CD94÷CD117+) and regulatory natural killer cells (CD34+CD56+CD94+CD117+).

In some embodiments, the actual percentage of the T cell subgroup includes an actual percentage of the naive helper T cells, an actual percentage of the aging helper T cells, an actual percentage of the regulatory helper T cells, an actual percentage of the naive cytotoxic T cells, an actual percentage of the aging cytotoxic T cells or a combination thereof. In some embodiments, the actual percentage of the natural killer cell subgroup includes an actual percentage of the cytotoxic natural killer cells, an actual percentage of the regulatory natural killer cells, or a combination thereof.

In some embodiments, the theoretical range of the T cell subgroup includes a theoretical range of the naive helper T cell, a theoretical range of the regulatory helper T cell, a theoretical range of the naive cytotoxic T cell, a theoretical range of the aging cytotoxic T cells. The theoretical range of the naive helper T cell is from 35% to 100%, the theoretical range of the regulatory helper T cell is from 3% to 10%, and the theoretical range of the naive cytotoxic T cell is from 20% to 100%. The theoretical range of the aging cytotoxic T cells is from 0% to 30%

In some embodiments, the theoretical range of the nature killer cell subgroup includes a theoretical range of the regulatory natural killer cell, in which the theoretical range of the regulatory natural killer cell is from 0% to 10%.

In some embodiments, the naive helper T cells and the naive killer T cells can provide the immune system with protection against the cancer cells. Therefore, once the actual percentage of the naive helper T cells or the actual percentage of the naive killer T cells is lower than the corresponding theoretical range, the cytotoxicity ability of the immune system of the individual against the cancers is less effective. The immune efficacy of the individual can be restored to a healthy state by administering the naive helper T cells or the naive cytotoxic T cells that the individual lacks, so as to enhance the effect of the immune system against cancer.

In some embodiments, the regulatory helper T cells are used to suppress other lymphocytes (e.g., other T cell subgroups) to balance immune system efficacy. Therefore, when the actual percentage of the regulatory helper T cells is higher than the theoretical range of the regulatory helper T cells, other lymphocytes will be excessively suppressed, resulting in poor immune efficacy and increase of the mortality rate of the individual suffering from the cancer. The immune efficacy of the individual can be restored to a healthy state by removing the excessive regulatory helper T cells of the individual, so as to enhance the effect of the immune system against cancer.

In some embodiments, the aging cytotoxic T cells represent the aging state of the individual's immune system. Therefore, if the actual percentage of the aging toxic T cells is higher than the theoretical range of the aging cytotoxic T cells, it is represented that the immune system is aging or declining. The immune efficacy of the individual can be restored to a healthy state by removing the excessive aging cytotoxic T cells of the individual, so as to enhance the effect of the immune system against cancer.

In some embodiments, the regulatory helper nature killer cells are used to suppress other lymphocytes (e.g., cytotoxic nature killer cells) to balance immune system efficacy. Therefore, when the actual percentage of the regulatory nature killer cells is higher than the theoretical range of the regulatory nature killer cells, other lymphocytes will be excessively suppressed, resulting in poor immune efficacy and increase of the mortality rate of the individual suffering from the cancer. The immune efficacy of the individual can be restored to a healthy state by removing the excessive regulatory nature killer cells of the individual, so as to enhance the effect of the immune system against cancer.

In some embodiments, the method further includes: (a) classifying the plurality of lymphocytes into a plurality of T cell subgroups based on expression or not of a plurality of first antigens, in which the plurality of T cell subgroups include a first T cell subgroup and a second T cell subgroup; (b) generating a deviation value of a ratio of T cell subgroups between an actual value of a ratio of two kinds of T cell subgroups and a theoretical range of the ratio of two kinds of T cell subgroups, in which the actual value of the ratio of two kinds of T cell subgroups is a quantitative ratio of the first T cell subgroup and the second T cell subgroup; and (c) administering the pharmaceutical composition including the first T cell subgroup or the second T cell subgroup to the individual based on the deviation value of the ratio of the T cell subgroups to allow the actual value of the ratio of two kinds of T cell subgroups of the individual to fall within the theoretical range of the ratio of two kinds of T cell subgroups.

In some embodiments, the theoretical range of the ratio of two kinds of T cell subgroups is derived from physiological values of a healthy population, in which the healthy population is human individuals with ages from 5 to 85 and does not suffer from immunodeficiency syndrome and hyperimmune syndrome.

In some embodiments, the theoretical range of the ratio of two kinds of T cell subgroups is from 1:1 to 5:1 when the first T cell subgroup is CD4 positive T cells and the second T cell subgroup is CD8 positive T cells. It can be understood that the CD4 positive T cells can activate the naive killer T cells. Therefore, when the actual value of the ratio of CD4/CD8 is lower than 1:1, the individual is in a state of hypoimmunity. A pharmaceutical composition including the CD4 positive T cells can be administered to the individual. In addition, an appropriate dose of the CD4 positive T cells to be administered is generated according to the blood volume of the individual, the actual number of the CD4 positive cells (the content of the CD4 positive cells in blood) and the corresponding theoretical range.

In some embodiments, the method further includes performing an immune cell therapy on the individual after the step of administering the pharmaceutical composition including the plurality of T cell subgroups to the individual or administering the pharmaceutical composition including the plurality of nature killer cells to the individual. In some embodiments, the immune cell therapy includes a natural killer cell therapy, a cytokine-induced killer cell therapy, a γδ T cell therapy, a dendritic cell therapy, a tumor infiltrating lymphocyte therapy, a chimeric antigen receptor T cell therapy, or a combination thereof.

It is worth emphasizing that current immune cell therapy does not compare the deviations between the actual values of the immune analysis indexes (such as the actual number of the total number of T cells, the actual number of the total number of natural killer cells, the percentage of the T cell subgroup, the percentage of the natural killer cell subgroup, and the ratio of CD4/CD8, or the like) and the corresponding theoretical ranges in advance. There is no steps of reinfusion of the lymphocytes cultured in vitro (such as T cells, nature killer cells, specific T cell subgroup or specific nature killer cell subgroup) into the individual or removal of the lymphocytes (such as T cells, nature killer cells, specific T cell subgroup or specific nature killer cell subgroup) from the individual according to the deviations of the abovementioned immune analysis indexes, thereby adjusting the actual values of the immune analysis indexes to the theoretical range. Therefore, the current immune cell therapy will have a huge gap in effectiveness due to difference of the immune system efficacy of the individuals (such as hypoimmunity or autoimmune diseases).

Relatively, according to the use of the present disclosure, it is ensured that the immune efficacy of the individual returns to a good state before performing the immune cell therapy according to the use of the present disclosure, so as to improve the success rate of the immune cell therapy.

For example, the immune efficacy of the T cells can be regulated to a healthy state by adjusting the actual number of the total number of T cells to the theoretical range of the total number of T cells and by adjusting the actual value of the ratio of the T cell subgroups to the theoretical range of the ratio of the T cell subgroups according to the deviation value of the total number of the T cells and the deviation value of the T cell subgroups before performing T cell related γδ T cell therapy, dendritic cell therapy, tumor infiltrating lymphocyte therapy, chimeric antigen receptor T cell therapy, so as to improve the therapeutic effect of the immune cells.

In another example, the immune efficacy of the nature killer cells can be regulated to a healthy state by adjusting the actual number of the total number of nature killer cells to the theoretical range of the total number of nature killer cells and by adjusting the actual value of the ratio of the nature killer cell subgroups to the theoretical range of the ratio of the nature killer cell subgroups according to the deviation value of the total number of nature killer cells and the deviation value of the nature killer cell subgroups before performing nature killer cell related natural killer cell therapy or killer therapy induced by cytokines, so as to to improve the therapeutic effect of the immune cells.

In some embodiments, the plurality of lymphocytes are derived from the individual and obtained by culturing in vitro. For example, before the treatment is administrated to the individual, the specific lymphocytes are pre-selected and cryopreserved from autologous blood. Therefore, when a cancer treatment is required in the future, the lymphocytes can be directly thawed, screened, expanded and then cultured for transfusion back to the individual. Pre-cryopreservation of the specific lymphocytes can not only save the acquisition process of the lymphocytes required for treatment, but also avoid immune rejection among different individuals.

In some embodiments, the cancer includes colorectal cancer, ovarian cancer, prostate cancer, breast cancer, brain cancer, cervical cancer, bladder cancer, anal cancer, uterine cancer, colon cancer, liver cancer, pancreatic cancer, lung cancer, endometrial cancer, bone cancer, testicular cancer, skin cancer, kidney cancer, stomach cancer, esophagus cancer, head and neck cancer, salivary gland cancer, hepatocellular carcinoma, non-small cell lung cancer, head and neck squamous cell cancer, basal cell cancer, cutaneous squamous cell cancer, cholangiocarcinoma, merkel cell carcinoma or a combination thereof.

In some embodiments of the present disclosure, a method of treating a cancer of an individual by a plurality of nature killer cells is provided, in which the method includes: (a) determining an actual value of a natural killer cell cytotoxicity of the plurality of natural killer cells, in which the actual value of the natural killer cell cytotoxicity is generated by following steps: co-culturing the plurality of natural killer cells with a plurality of cancer cells with a quantitative ratio; and generating a quantity percentage of the cancer cells that die to obtain the actual value of the natural killer cell cytotoxicity; (b) generating a deviation value of a natural killer cell cytotoxicity between an actual value of the natural killer cell cytotoxicity and a theoretical range of the natural killer cell cytotoxicity based on the actual value of the natural killer cell cytotoxicity and the theoretical range of the natural killer cell cytotoxicity; (c) administering the pharmaceutical composition including the plurality of nature killer cells to the individual based on the deviation value of the natural killer cell cytotoxicity to allow the actual value of the natural killer cell cytotoxicity of the individual to fall within the theoretical range of the natural killer cell cytotoxicity.

It is understood that the individual suffering from a cancer often has insufficient immunity or excessive autoimmunity due to insufficient actual number of the total number of nature killer cells, excessive actual number of the total number of nature killer cells, or insufficient natural killer cell cytotoxicity. The present disclosure regulates the performance of the individual's immune system to a healthy state by adjusting the actual value of the natural killer cell cytotoxicity of nature killer cells to the theoretical range (such as adjusting the actual number of the total number of nature killer cells to the theoretical range, or activating the nature killer cells in the individual), and the individual's immune system against the cancer is increased, thereby improving the therapeutic effect of the cancer. Compared with traditional radiation therapy or chemotherapy, the use of the present disclosure will not cause damage to the individual's cells. In addition, the use of this disclosure can also be used in combination with other immune cell therapies, the effectiveness of immune cell therapy is increased through precise regulation of the natural killer cell cytotoxicity of nature killer cells.

In some embodiments, the theoretical range of the natural killer cell cytotoxicity is derived from physiological values of a healthy population, in which the healthy population is human individuals with ages from 5 to 85 and does not suffer from immunodeficiency syndrome and hyperimmune syndrome.

In some embodiments, the theoretical range of the natural killer cell cytotoxicity is from 0% to 58.8% when the quantitative ratio is 6.25, the theoretical range of the natural killer cell cytotoxicity is from 1.7% to 88.7% when the quantitative ratio is 12.5, the theoretical range of the natural killer cell cytotoxicity is from 17.4% to 100% when the quantitative ratio is 25, and the theoretical range of the natural killer cell cytotoxicity is from 35.3% to 100% when the quantitative ratio is 50.

In some embodiments, at the step of (a), the theoretical range of the natural killer cell cytotoxicity is 57.4% to 100% when the quantitative ratio is 50, and interleukin-2 with an action concentration of 100 international units/mL is added to co-culture with the plurality of natural killer cells and the cancer cells. It is worth noting that at the action concentration, IL-2 can completely inhibit the activity of the regulatory natural killer cells (and the regulatory natural killer cells can inhibit the activity of the cytotoxic natural killer cells). Therefore, the actual value of the natural killer cell cytotoxicity obtained under this condition reflects the activity of the cytotoxic natural killer cells of the individual, i.e., the maximum activity of the natural killer cells.

In some embodiments, the method further includes: generating a deviation value of a total number of nature killer cells between an actual number of the total number of nature killer cells and a theoretical range of the total number of nature killer cells; and administering the pharmaceutical composition including the plurality of nature killer cells to the individual based on the deviation value of the natural killer cell cytotoxicity and the deviation value of the total number of nature killer cells to allow the actual number of the total number of nature killer cells of the individual to fall within the theoretical range of the total number of nature killer cells, thereby allowing the actual value of the natural killer cell cytotoxicity to fall within the theoretical range of the natural killer cell cytotoxicity.

In some embodiments, the method further includes: classifying the plurality of nature killer cells into a plurality of natural killer cell subgroups based on expression or not of a plurality of antigens, in which the plurality of natural killer cell subgroups include a regulatory natural killer cell, in which the regulatory natural killer cell is CD34+, CD56+, CD94+, and CD117+; generating a deviation value of a regulatory natural killer cell between an actual percentage of the regulatory natural killer cell and a theoretical range of the regulatory natural killer cell, in which the actual percentage of the regulatory natural killer cell is an actual quantity percentage of the regulatory natural killer cell in lymphocytes; administering the pharmaceutical composition including the regulatory natural killer cell to the individual based on the deviation value of the natural killer cell cytotoxicity and the deviation value of the regulatory natural killer cell to allow the actual percentage of the regulatory natural killer cell of the individual to fall within the theoretical range of the regulatory natural killer cell, thereby allowing the actual value of the natural killer cell cytotoxicity to fall within the theoretical range of the natural killer cell cytotoxicity.

It can be understood that, the profile of the immune efficacy of the natural killer cells is presented by analyzing the trend of the natural killer cell cytotoxicity in different quantitative ratios and the natural killer cell cytotoxicity measured under the condition that the quantitative ratio is 50 and IL-2 is added (the maximum natural killer cell activity).

For example, if the quantitative ratio of the natural killer cells and the cancer cells co-cultured increases, but the actual value of the natural killer cell cytotoxicity of each group is lower than the theoretical range, and the actual value of the natural killer cell cytotoxicity of the group with the maximum activity of the natural killer cells (IL-2 control group) is also lower than the theoretical range, it is represented that a deviation of the natural killer cell cytotoxicity exists between the actual value of the natural killer cell cytotoxicity and the theoretical range of the natural killer cell cytotoxicity.

Furthermore, the actual number of the total number of natural killer cells can be further analyzed. When the actual number of the total number natural killer cells is lower than the corresponding theoretical range, the natural killer cells can be administered to the individual to allow the actual number of the total number of natural killer cells to fall within the theoretical range of the total number of natural killer cells, so that the actual value of the natural killer cell cytotoxicity falls within the theoretical range of the natural killer cell cytotoxicity. On the other hand, the actual percentage of regulatory natural killer cells can also be analyzed simultaneously. When the actual percentage of regulatory natural killer cells is higher than the corresponding theoretical range, it is represented that the efficacy of natural killer cells is over-suppressed. The actual percentage of regulatory natural killer cells can fall within the corresponding theoretical range by removing the excessively high ratio of regulatory natural killer cells, thereby restoring the actual value of the natural killer cell cytotoxicity to the theoretical range of the natural killer cell cytotoxicity.

In another example, if the quantitative ratio increases, the actual value of the natural killer cell cytotoxicity of each group is lower than the theoretical range, but the actual value of the natural killer cell cytotoxicity of the group with the maximum activity of the natural killer cells (IL-2 control group) falls within the theoretical range, it is represented that the activity of the nature killer cells is insufficient. The immune efficacy can be increased by isolating the nature killer cells from the individual, activating and culturing the nature killer cells by IL-2, confirming the actual value of the natural killer cell cytotoxicity of the nature killer cells activated is restored to the corresponding theoretical range, and then reinfusing the nature killer cells into the individual.

In some embodiments, the method further includes performing an immune cell therapy on the individual after the step of administering the pharmaceutical composition including the plurality of nature killer cell subgroups to the individual or administering the pharmaceutical composition including the plurality of nature killer cells to the individual. In some embodiments, the immune cell therapy includes a natural killer cell therapy, a cytokine-induced killer cell therapy, a γδ T cell therapy, a dendritic cell therapy, a tumor infiltrating lymphocyte therapy, a chimeric antigen receptor T cell therapy, or a combination thereof.

It is worth emphasizing that the current immune cell therapy will have a huge gap in effectiveness due to difference of the immune system efficacy of the individuals (such as hypoimmunity or autoimmune diseases). Relatively, according to the use of the present disclosure, it is ensured that the natural killer cell cytotoxicity of the individual returns to a good state before performing the immune cell therapy, so as to improve the success rate of the immune cell therapy.

For example, the actual number of the total number of nature killer cells is adjusted to the theoretical range of the total number of nature killer cells, the actual value of the ratio of the nature killer cell subgroups is adjusted to the theoretical range of the ratio of the nature killer cell subgroups, or both are performed simultaneously according to the deviation value of the natural killer cell cytotoxicity and according to the deviation value of the total number of nature killer cells, the deviation value of the regulatory nature killer cells or both before performing nature killer cell therapy, so as to allow the actual value of the natural killer cell cytotoxicity to fall within the theoretical range of the natural killer cell cytotoxicity.

In some embodiments, the plurality of nature killer cells are derived from the individual and obtained by culturing in vitro. For example, before the treatment is administrated to the individual, the specific lymphocytes are pre-selected and cryopreserved from autologous blood. Therefore, when a cancer treatment is required in the future, the lymphocytes can be directly thawed, screened, expanded and then cultured for transfusion back to the individual. Pre-cryopreservation of the specific lymphocytes can not only save the acquisition process of the nature killer cells required for treatment, but also avoid immune rejection among different individuals.

In some embodiments, the cancer includes colorectal cancer, ovarian cancer, prostate cancer, breast cancer, brain cancer, cervical cancer, bladder cancer, anal cancer, uterine cancer, colon cancer, liver cancer, pancreatic cancer, lung cancer, endometrial cancer, bone cancer, testicular cancer, skin cancer, kidney cancer, stomach cancer, esophagus cancer, head and neck cancer, salivary gland cancer, hepatocellular carcinoma, non-small cell lung cancer, head and neck squamous cell cancer, basal cell cancer, cutaneous squamous cell cancer, cholangiocarcinoma, merkel cell carcinoma or a combination thereof.

To further illustrate the method and system for detecting the immune efficacy and use of lymphocytes for preparing a pharmaceutical composition for treating a cancer of an individual (a method of treating the cancer by adjusting the content of the lymphocyte) provided by various embodiments of the present disclosure, following implementations were performed. It should be noted that the following embodiments are provided for exemplary purposes only, and are not intended to limit the present disclosure.

I. Method and System for Detecting Immune Efficacy

1. Detection of Types of Immune Cells and State of Antigen Expression (Detecting Actual Values of Immune Analysis Indexes of T Cells and Nature Killer Cells)

First, 100 μL of a whole blood sample from human was added to a test tube containing an appropriate concentration of anticoagulant, in which the anticoagulant could be heparin or ethylenediaminetetraacetic acid (EDTA). If EDTA was added, an acting weight percentage of EDTA was 0.1%.

According to antigens (classification markers) exemplified in Table 1, a specific antibody with a fluorescent group (e.g., fluorescein isothiocyanate (FITC)) was selected, and immunostaining was performed with a suitable method according to locations of the antigens (e.g., cell surface antigens or intracellular antigens) to target specific cells to classify the cells into a plurality of cell subgroups.

TABLE 1
Classification Markers of Cell Subgroups

| | | Marker 1 | Marker 2 | Marker 3 | Marker 4 | Marker 5 |
|---|---|---|---|---|---|---|
| T cells | Naive Helper Type | CD3 | CD4 | CD45RA | CD62L | CD28 |
| | Aging Helper Type | CD3 | CD4 | CD45RO | CD62L | |
| | Regulatory Helper Type | CD3 | CD4 | CD25 | FoxP3 | CD39 |
| | Naive Cytotoxic Type | CD8 | CD27 | CD45RA | CD62L | CD127 |
| | Aging Cytotoxic Type | CD8 | CD27 | CD45RO | CD62L | CD57 |
| Natural | Cytotoxic Type | CD16 | CD34 | CD56 | CD94 | CD117 |
| Killer | | | | | | |
| Cells | Regulatory Type | CD34 | CD56 | CD94 | CD117 | |

If the selected antigens were the cell surface antigens (e.g., CD3, CD4, CD8, CD25, CD27, CD34, CD39, CD45RO, CD45RA, CD56, CD57, CD62L, CD94, CD117, and CD127), following surface antigen staining steps were performed.

100 μL of an antibody labeled with a fluorescent group (e.g., Invitrogen's Flow Cytometry Antibodies) was added into a test tube containing 100 μL of the whole blood sample, so that a volume ratio of the whole blood sample to the antibody was 1:1, and an antibody dilution ratio was 50 to 100-fold. Next, it was rotated in the dark for 30 minutes to 1 hour at a temperature of 2° C. to 8° C. Next, cells in the whole blood sample were washed with a flow cytometry staining liquid (Invitrogen eBioscience Flow Cytometry Staining Buffer, product number 00-4222-26) to obtain a first cell staining liquid.

If the selected antigens included the surface antigens and the intracellular antigens (e.g., FoxP3), following steps were performed after the aforementioned surface antigen staining steps were performed.

2 mL of fix/lyse liquid (Invitrogen eBioscience 1-Step Fix/Lyse Solution (10×), product number 00-5333-54) was added to 100 μL of the first cell staining liquid at room temperature and inverted to mix and to lyse red blood cells to avoid the red blood cells interfering with subsequent flow cytometry analysis. Next, it was cultured in the dark for 15 minutes to 60 minutes at room temperature to obtain a treatment liquid.

The treatment liquid was centrifuged at 500×g for 5 minutes at room temperature, and the cells were pelleted, and a supernatant was removed.

Under dark conditions, 2 mL of permeabilization liquid (prepared by 10-fold dilution of Invitrogen eBioscience Permeabilization Buffer (10×) (product number 00-8333-56)) was added to suspend the cells. Next, centrifugation was performed at 500 xg for 5 minutes at room temperature, and a supernatant was removed, and steps of adding the permeabilization liquid, centrifuging and removing the supernatant were repeated once more.

The cells were suspended with 100 μL of the flow cytometry staining liquid, and 100 μL of the antibody labeled with the fluorescent group (e.g., Invitrogen's flow cytometry antibodies) was then added, so that an antibody dilution ratio was 50 to 100-fold. Next, it was rotated in the dark for 20 to 60 minutes at room temperature. The cells were washed with 2 mL of the flow cytometry staining liquid, and then centrifuged, and a supernatant was removed, and the cells were suspended with 500 μL of the flow cytometry staining liquid to obtain a second cell staining liquid.

Sample cell surface antigen data were obtained using a flow cytometer according to expression or not of the cell classification markers (see Table 1) to classify T cell subgroups and natural killer cell subgroups, and contents of T cells and nature killer cells in a whole blood sample (actual number of total number of T cells and actual number of total number of nature killer cells), ratios of the T cell subgroups and the natural killer cell subgroups in lymphocytes respectively (an actual percentage of a T cell subgroup and an actual percentage of a nature killer cell subgroup) and a ratio of CD4 positive cells/CD8 positive cells were generated. In addition, if required, a proportion of T cells with a specific marker antigen (e.g., CD28) in all the T cells could be analyzed.

It's worth emphasizing that the classification markers used in this application are confirmed multiple times by using the exclusive antigens of each T cell subgroup and each natural killer cell subgroup to further improve the accuracy of classification.

In detail:

Regarding naive helper T cells, in addition to the use of CD45RA (marker of naive T cells) and CD28 (marker of naive helper T cells) for double conformation, CD62L (marker of non-regulatory helper T cells) is used for excluding regulatory helper T cells to reduce the probability of regulatory helper T cells being mixed.

Regarding aging helper T cells, in addition to the use of CD45RO (marker of aging T cells) for conformation, CD62L is used for reducing the probability of regulatory helper T cells being mixed.

Regarding regulatory helper T cells, CD25, FoxP3 and CD39 are used for triple conformations of the exclusive antigens of the regulatory helper T cells, thereby increasing a correct ratio of grouping of the regulatory helper T cells.

Regarding naive cytotoxic T cells, in addition to the use of CD45RA of the marker of the naïve cells for classifying the naive T cells from the cytotoxic T cells (CD8), CD127, the exclusive antigens of the naive cytotoxic T cells, is further used for double conformation to increase a correct ratio of grouping of the naive cytotoxic T cells.

Regarding aging cytotoxic T cells, in addition to the use of CD45RO of the marker of the aging cells for classifying the aging T cells from the cytotoxic T cells (CD8), CD57, the exclusive antigens of the aging cytotoxic T cells, is further used for double conformation to increase a correct ratio of grouping of the aging cytotoxic T cells.

Regarding cytotoxic nature killer cells and regulatory nature killer cells, both are classified by CD16 (marker of cytotoxic nature killer cells) and confirmed multiple times through CD34, CD56, CD94 and CD117 of the exclusive antigens of the nature killer cells.

2. Detection of Actual Value of Natural Killer Cell Cytotoxicity

The natural killer cells are the first line of defense against viruses and cancer. Therefore, the detection of the natural killer cell cytotoxicity can be used to evaluate innate immunity against viruses and cancer. The detection of the natural killer cell cytotoxicity included following steps.

First, the natural killer cells (e.g., according to Table 1 of 1., the appropriate antibodies were selected and isolated from the whole blood sample) and K-562 cells were co-cultured with different quantitative ratios (effector cell/target cell=ET ratio, for example, natural killer cells/K-562 cells=6.25, 12.5, 25, 50), and a proportion of K-562 cells that died was analyzed by flow cytometer, so as to evaluate the natural killer cell cytotoxicity to determine health and cancer resistance of the natural killer cells.

In addition, since the regulatory natural killer cells could inhibit activity of the cytotoxic natural killer cells, IL-2 (action concentration: 100 international units/mL=100 IU/mL) were further added under a condition that the E:T ratio was 50 to inhibit activity of the regulatory natural killer cells, and to evaluate activity of the cytotoxic natural killer cells by comparing with a group without adding IL-2, to analyze maximum activity of the natural killer cells and to determine whether the natural killer cells were in a state of immunosuppression.

Specifically, as the ET ratio increased from 6.25 to 50, the quantity percentage of K-562 cells that died (or the count of K-562 cells that died) showed an increase in arithmetic progression, which represented that the natural killer cells were healthy and energetic and had ability to clear the cancer cells. Conversely, as the ET ratio continues to increase, the quantity percentage of K-562 cells that died (or the count of K-562 cells that died) was lower than a reference range and did not show an increase in arithmetic progression, which represented that the natural killer cells (especially the cytotoxic natural killer cells) were unhealthy and lack of anti-virus and anti-cancer ability.

To further clarify the state of the natural killer cells, the quantity percentage of K-562 cells that died and the count of the regulatory natural killer cells in the IL-2 control group could be simultaneously referenced.

If in the IL-2 control group, the quantity percentage of K-562 cells that died was lower than the reference range, and the proportion of the regulatory natural killer cells was high, it represented an autoimmune suppression state (common causes included suffering from cancer or chronic viral infection). On the contrary, if the proportion of the regulatory natural killer cells was normal, it represented an immunocompromised state (common causes included aging (especially over 65 years old), drug effects (e.g., immunosuppressive or chemotherapy, etc.), radiation therapy or have been exposed to excessive radiation damage).

In addition, if the quantity percentage of K-562 cells that died in the IL-2 control group fell within the reference range, it meant that the natural killer cells could still be stimulated and activated to generate anti-cancer and anti-virus ability, that is, although original activity of the natural killer cells was worse than that of ordinary people, but did not affect physical health.

Therefore, by testing the natural killer cell cytotoxicity, the anti-cancer and anti-virus ability and health of the natural killer cells could be effectively quantitatively evaluated, and a more accurate analysis could be provided for clinical disease diagnosis.

3. Analysis of Immune Efficacy

Through the aforementioned 1, the types of the immune cells (the T cell subgroups and the natural killer cell subgroups) and the state of the antigen expression in the whole blood sample were obtained, and the actual percentage of each of the cell subgroups (the T cell subgroups and the natural killer cell subgroups) in the lymphocytes were calculated. After the actual value of the natural killer cell cytotoxicity was obtained through the aforementioned I.2., immune efficacy was judged according to following Tables 2 to 4. When all of the actual values fell within the reference ranges (e.g., within the theoretical ranges or the tolerance ranges), it was judged that immune efficacy was normal. Further, when the actual value (e.g., the actual percentage) of each of the cell subgroups met the theoretical range, it was judged that immune efficacy was good. However, when any of the actual values (e.g., the actual percentages) did not meet the reference range (i.e., did not meet the theoretical range and the tolerance range), it was judged that immune efficacy was poor.

TABLE 2
Immune Analysis Index of T cells

| Index | Description | Reference Range |
|---|---|---|
| Total Number of T | content of T cells in blood | from 700 cells/μL to 2500 |
| cells | | cells/μL |
| | | (the same as the Theoretical |
| | | Range) |
| Ratio of CD4/CD8 | ratio of regulatory T cells and cytotoxic T | from 1:1 to 5:1 |
| | cells | (the same as the Theoretical |
| | | Range) |
| Quantity of CD4 | content of regulatory T cell in blood | ≥400 cells/μL |
| positive cells | | (the same as Theoretical |
| | | Range) |
| Quantity of CD8 | content of cytotoxic T cell in blood | ≥200 cells/μL |
| positive cells | | (the same as the Theoretical |
| | | Range) |
| Ratio of naive | Naive helper T cells can differentiate into | Theoretical range: ≥35% |
| helper T cells | other T cells, which can provide | 20% ≤ Tolerance range < 35% |
| | protection against new pathogens and | |
| | reflect activity and protective ability of | |
| | the immune system | |
| Ratio of CD28 | Reduced in autoimmune diseases | Theoretical range: ≥90% |
| positive (CD28+) in | | 80% ≤ Tolerance range < 90% |
| helper T cells | | |
| (CD28+ are naive | | |
| helper T cells) | | |
| Ratio of regulatory | Regulatory helper T cells maintain | Theoretical range: 3%-10% |
| helper T cells | immune system balance and inhibit other | 1% ≤ Tolerance range < 3%/ |
| | immune cells to avoid immune cell | 10% < Tolerance range ≤ 15% |
| | overreaction. However, if excessive, it | |
| | will inhibit the body's anti-cancer immune | |
| | defense function and increase cancer | |
| | mortality | |
| | High: lung cancer, pancreatic cancer, | |
| | breast cancer | |
| | Low: multiple sclerosis, rheumatoid | |
| | arthritis, type 1 diabetes | |
| Ratio of naive | Naive cytotoxic T cells can differentiate | Theoretical range: ≥20% |
| cytotoxic T cells | into other T cells, which can provide | 15% ≤ Tolerance range < 20% |
| | protection against new pathogens and | |
| | reflect activity and protective ability of | |
| | the immune system | |
| Ratio of aging | The change of the surface antigen CD57 | Theoretical range ≤30% |
| cytotoxic T cells | from negative (CD57−) to positive | 30% < Tolerance range ≤ |
| | (CD57+, aging cytotoxic T cells) is an | 30%-50% |
| | indicator of T cell aging and apoptosis | |
| | High: Immune decline, cancer, | |
| | arteriosclerosis, cardiovascular disease, | |
| | autoimmune disease, alcoholism, | |
| | chronic disease | |

Note 1:
Ratio of CD4/CD8 refers to a ratio of CD4 positive T cells and CD8 positive T cells.
Note 2:
The ratios of the naive helper T cells, the ratio of the regulatory helper T cells, the ratio of the naive cytotoxic T cells and the ratio of the aging cytotoxic T cells in Table 2 represent the quantity percentages of the specific T cell subgroups in the lymphocytes.

TABLE 3
Immune Analysis Index of Natural Killer Cells

| Index | Description | Reference Range |
|---|---|---|
| Total number of | A low count of natural killer cells may | Theoretical range: from 100 |
| natural killer cells | be associated with a high risk of | cells/μL to 300 cells/μL |
| (count of natural killer | developing cancer. However, if the | 50 cells/μL ≤ Tolerance |
| cells per microliter of | count is too large or the activity is too | range < 100 cells/μL |
| the whole blood | strong, it may inhibit performance of | |
| sample) | other cells and thus reduce immune | |
| | efficacy | |
| Ratio of regulatory | Regulatory natural killer cells inhibit | Theoretical range: ≤10% |
| natural killer cells | activity of cytotoxic natural killer cells. | 10% < Tolerance range ≤ |
| | Representing an adverse immune | 20% |
| | response in cancer patients. | |
| | In addition, patients with chronic viral | |
| | infections are often overrepresented | |

Note 3:
The ratio of the regulatory natural killer cells in Table 3 represents the quantity percentage of the specific natural killer cell subgroup in the lymphocytes.

TABLE 4
Natural Killer Cell Cytotoxicity

| | Reference Range |
|---|---|
| | (the same as Theoretical Range) |
| | (quantity percentage |
| Immune Analysis Index | of K-562 cells that die) |
| E:T ratio 6.25 | Upper limit: 58.8% |
| | Lower limit: 0% |
| E:T ratio 12.5 | Upper limit: 88.7% |
| | Lower limit: 1.7% |
| E:T ratio 25 | Upper limit: 100% |
| | Lower limit: 17.4% |
| E:T ratio 50 | Upper limit: 100% |
| | Lower limit: 35.3% |
| Maximum activity of natural killer | Upper limit: 100% |
| cells (IL-2 control group, in which | Lower limit: 57.4% |
| action concentration of IL-2 is 100 | |
| IU/mL, and E:T ratio is 50) | |

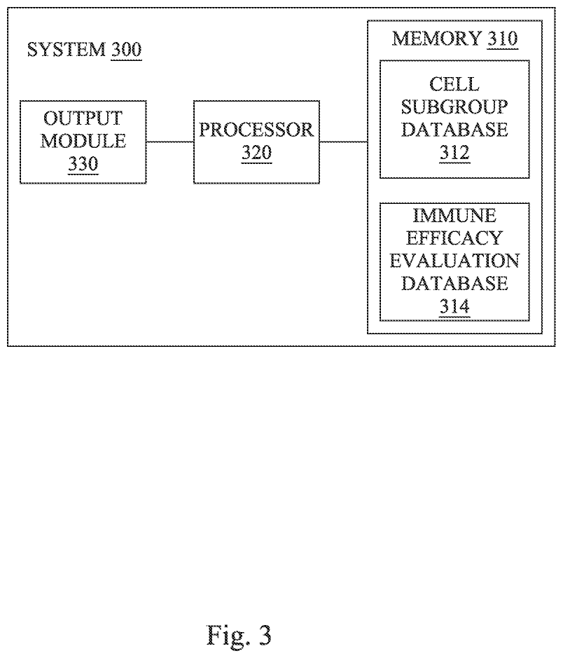

In addition, the method for analyzing immune efficacy can also be built into a system 300 specifically. Referring to FIG. 3, the system 300 may include a memory 310, a processor 320 and an output module 330.

First, electronic devices such as personal computers, smart phones, and servers can be used to transfer the sample cell surface antigen data (including the antigen expression of the cells in the whole blood sample) obtained by the flow cytometry analysis in 1. “Detection of Types of Immune Cells and State of Antigen Expression” and the natural killer cell cytotoxicity data obtained in 2. “Detection of Natural Killer Cell Cytotoxicity” in a file format to the memory 310 (e.g., read-only memory, flash memory, disk or cloud database, etc.) for storage.

The system 300 also includes a processor 320 connected to the memory 310. The processor 320 includes a central processor, an image processor, a microprocessor, etc., such as a processing unit including multi-core or a combination of a plurality of processing units. The processor 320 can access the sample cell surface antigen data and the natural killer cell cytotoxicity data in the memory 310 and perform following analysis.

First, the processor 320 analyzes the sample cell surface antigen data according to the cell subgroup database 312 (including the T cell subgroup classification information and the natural killer cell subgroup classification information in Table 1), and classifies the cells in the whole blood sample into cell subgroups (including T cell subgroups and natural killer cell subgroups), and calculates a quantity percentage of each of the cell subgroups in lymphocytes to obtain T cell subgroup count data and natural killer cell subgroup count data.

Next, the processor 320 analyzes the T cell subgroup count data, the natural killer cell subgroup count data, and the natural killer cell cytotoxicity data according to an immune efficacy evaluation database 314 (including the T cell subgroup reference range data, the natural killer cell subgroup reference range data and the natural killer cell cytotoxicity reference range data in Tables 2 to 4) to generate immune efficacy data.

The system 300 further includes the output module 330 connected to the processor 320, and the output module 330 outputs the immune efficacy data as an immune efficacy report for presentation on a display screen (not shown) or to another electronic device, to evaluate whether to cryopreserve specific T cell subgroups or specific natural killer cell subgroups.

II. Isolation and Cryopreservation of Target Cells

According to the analysis results of I.3., it was determined whether to cryopreserve specific T cell subgroups or specific natural killer cell subgroups, for example, when the actual percentage of the naive helper T cells met the naive helper T cell reference range, the naive helper T cells were cryopreserved.

1. Isolation of Target Cells

The target cells (the specific T cell subgroup or the specific natural killer cell subgroup) were isolated by the immunomagnetic bead cell sorting method, in which the immunomagnetic bead cell sorting method included a positive sorting system and a negative sorting system.

A sorting process of the positive sorting system (using invitrogen's Dynal® positive isolation kits and FlowComp™ kits series products) included: an antibody with specially modified biotin (Antibody Mix in FlowComp™ kit series products) was used to label the target cells, and positive sorting magnetic beads with streptavidin (FlowComp™ Dynabeads™ in FlowComp™ kit series) were then added to bind streptavidin on the positive sorting magnetic beads to biotin, and the cells bound to the positive sorting magnetic beads were magnetically sorted, and the positive sorting magnetic beads bound to the cells were removed to obtain the target cells.

A sorting process of the negative sorting system (using Invitrogen's Negative isolation (Untouched™) kits) included: antibody mixtures with biotin (Antibody mix in Negative isolation (Untouched™) kits) were used to label cells to be removed, and negative sorting magnetic beads with streptavidin (Depletion MyOne™ SA Dynabeads® in Negative isolation (Untouched™) kits) were then added to bind streptavidin on the negative sorting magnetic beads to biotin, and the cells to be removed were then removed by magnetic screening, and the target cells that were not bound to the negative sorting magnetic beads were sorted out.

The following exemplifies manners in which the T cell subgroups and the natural killer cell subgroups were respectively sorted.

(1) Regulatory Helper T Cells (CD3+CD4+CD25+FoxP3+CD39+)

CD3+CD4+ was used to screen out helper T cells (including naive helper T cells, aging helper T cells, and regulatory helper T cells), and CD25+CD45− was used to screen out the regulatory helper T cells.

The sorting process of CD3+ cells was as follows (taking the positive sorting system as an example).

First, peripheral blood mononuclear cells (PBMCs) were separated from a whole blood sample, and a separation liquid was used to prepare PBMCs into a PBMC solution with cell density of 5×10⁶ cells/mL to be separated, in which the separation liquid was a phosphate buffer without calcium ions and magnesium ions, which was supplemented with 0.1% bovine serum albumin (BSA) and 2 mM of ethylenediaminetetraacetic acid (EDTA) (purchased from Gibco, Inc., product No. 14190).

Next, cells expressing CD3 were isolated using invitrogen's Dynabeads® FlowComp™ Human CD3 kit (product No. 113-65D), which specifically included following steps.

25 μL of CD3 antibody with specially modified biotin (Antibody Mix in the kit) and 500 μL of PBMC solution to be separated were mixed and reacted at 2° C. to 8° C. for 10 minutes to make the CD3 antibody bind to the cells expressing CD3, and the PBMCs were washed with 2 mL of the separation liquid, and centrifugation was performed at 350 xg for 8 minutes, and a supernatant was removed.

Next, after the PBMCs was suspended with 1 mL of the separation liquid, 75 μL of the positive sorting magnetic beads (FlowComp™ Dynabeads™ in the kit) was added and cultured at room temperature (about 25° C.) with rotation for 15 minutes. A positive mixture was obtained.

Next, the positive mixture was placed on a magnet for at least 1 minute, and a supernatant was removed to retain magnetic bead-labeled cells. After the magnetic bead-labeled cells were removed from the magnet, 1 mL of the separation liquid was added to suspend the magnetic bead-labeled cells. The magnetic bead-labeled cells were placed on the magnet again for at least 1 minute, and a supernatant was removed.

Next, 1 mL of a magnetic bead release liquid (FlowComp™ Release Buffer in the kit) was added and rotated for 10 minutes at room temperature. After the magnetic bead release liquid containing the magnetic bead-labeled cells was placed on the magnet for 1 minute, a supernatant (containing the cells expressing CD3) was transferred to a new centrifuge tube, and the cells were pelleted by centrifugation at 350×g for 8 minutes, and a supernatant was removed to obtain the cells expressing CD3.

Next, a corresponding kit was selected and steps similar to those described above were performed to further sort CD4+ cells (CD3+CD4+ cells) from CD3+ cells, and then further sort CD25+ cells (CD3+CD4+CD25+ cells) from CD3+CD4+ cells, and then further sort and remove CD45+ cells to obtain CD45− cells (CD3+CD4+CD25+CD45− cells) to obtain the regulatory helper T cells.

(2) Naive Helper T Cells (CD3+CD4+CD45RA+CD62L+CD28+) or Aging Helper T Cells (CD3+CD4+CD45RO+CD62L+CD28−)

Referring to the specific sorting process of the aforementioned CD3+ cells, CD3+CD4+ was used to screen out the helper T cells (including the naive helper T cells, the aging helper T cells, and the regulatory helper T cells), and CD25−CD45+(CD45+ here was used to at least screen out cells expressing CD45RA or CD45RO, in which CD45RA and CD45RO are different isomers of CD45) was then used to screen out the naive helper T cells and the aging helper T cells. Finally, CD28+ or CD28− was used to screen out the naive helper T cells (CD28-F) or the aging helper T cells (CD28−).

(3) Naive Cytotoxic T Cells or Aging Cytotoxic T Cells

Referring to the specific sorting process of the aforementioned CD3+ cells, CD3+CD8+ was used to screen out the cytotoxic T cells (including the naive cytotoxic T cells and the aging cytotoxic T cells), and CD57− or CD57+ was then used to screen out the naive cytotoxic T cells (CD57−) or the aging cytotoxic T cells (CD57+).

(4) Cytotoxic Natural Killer Cells or Regulatory Natural Killer Cells

Referring to the specific sorting process of the aforementioned CD3+ cells, CD34+CD56+ was used to screen out the natural killer cell subgroups (including the cytotoxic natural killer cells and the regulatory natural killer cells), and CD16+ or CD16− was then used to screen out the cytotoxic natural killer cells (CD16+) or the regulatory natural killer cells (CD16−).

(5) Isolation of Each of T Cell Subgroups and Nature Killer Cell Subgroups in One Blood Sample

The classification markers used in this application can sequentially separate each T cell subgroup and each natural killer cell subgroup from the same blood sample, without respectively screening each cell subgroup by dividing the blood sample into multiple blood samples, which not only saves screening time and consumables, improves screening efficiency, and reduces quantification errors. In addition, the selection of markers in the application allows the blood sample to be screened for two antigens at one time, so that the quantity of the isolations can be controlled within the acceptable range of cells (2-3 isolation steps) to avoid damage to cells.

Therefore, the markers selected in this application can improve the applicability and the accuracy of clinical testing. Please refer to the following process for details:

Regarding to T Cells:

(1) Helper T Cells:

First of all, CD3 and CD4 could be used to isolate helper T cells (CD3 is a T cell marker, CD4 is a helper T cell marker). Then, CD45RA (naive T cell marker) and CD62L (marker of non-regulatory helper T cells in T cells), CD45RO (marker of aging T cells) and CD62L, CD25 and FoxP3 (both are markers of regulatory helper T cells) were respectively selected to initially isolate naive helper T cells, aging helper T cells and regulatory helper T cells from the isolated cells. Finally, for the cells screened by expressing CD45RA and CD62L, or CD25 and FoxP3, CD28 (naive helper T cell marker) and CD39 (regulatory helper T cell marker) were further used to re-confirm the naive helper T cells and the regulatory helper T cells, so as to obtain the naive helper T cells, the aging helper T cells and the regulatory helper T cells.

(2) Cytotoxic T Cells:

CD8 and CD27 (both were cytotoxic T cell markers) could be used to isolate cytotoxic T cells from the remained cells after finishing screening the helper T cells. Then, CD45RA (naive T cell marker) and CD62L (marker of non-regulatory helper T cells in T cells), CD45RO (aging T cell marker) and CD62L were respectively selected to initially isolate naive cytotoxic T cells and aging cytotoxic T cells. Finally, CD127 (naive cytotoxic T cell marker) and CD57 (aging cytotoxic T cell marker) were further used to re-confirm the naive cytotoxic T cells and the aging cytotoxic T cells, so as to obtain the naive cytotoxic T cells and the aging cytotoxic T cells.

Regarding to Nature Killer Cells:

CD16 (cytotoxic nature killer cell marker) and CD34 (nature killer cell marker) could be used to isolate the cytotoxic nature killer cells from the cells not isolated after the isolation of the T cell subgroups. Then, the cytotoxic nature killer cell were confirmed multiple times sequentially by CD56 and CD94, CD117 (all of the three markers were the nature killer cells markers), so as to obtain cytotoxic nature killer cells. Then, regulatory nature killer cells were isolated from the remained cells sequentially by CD34 and CD56, CD94 and CD117.

2. Cryopreservation of Target Cells

The target cells sorted out in the aforementioned 4.1 “Isolation of Target Cells” were subcultured, and a culture medium containing the target cells (a corresponding culture medium was selected according to different cells) was centrifuged to pellet the cells, and a supernatant was removed, and the target cells suspended using a culture medium containing 5% by weight of dimethyl sulfoxide (DMSO), so that target cell density was 1×10⁶/mL to 5×10⁶/mL, and it was aliquoted into cryopreservation tubes for preservation, and those were stored at 4° C. for 10 minutes to 30 minutes, −20° C. for 30 minutes, −80° C. for 16 hours to 18 hours in sequence, and finally moved to liquid nitrogen for long-term cryopreservation, or placed in a programmed cooling machine (e.g., cooling rate of 1° C. to 3° C. per minute) to −80° C. and then placed in liquid nitrogen for long-term cryopreservation.

It should be emphasized that if it was stored at −20° C. for more than 1 hour, ice crystals would be too large and thus cell viability would be reduced. Additionally, the steps of 4° C. and −20° C. could be skipped, and those could be directly placed at −80° C., but cell viability would be reduced.

Since the T cell subgroups and the natural killer cell subgroups respectively have different functions and play different roles in the immune system (e.g. the naive helper T cells can help fight new pathogens, and the cytotoxic natural killer cells can suppress cancer), the present disclosure provides the method and system for detecting the immune efficacy by classifying the T cell subgroups (e.g., classifying the naive helper T cells expressing CD28) via first antigens including CD28, and classifying the natural killer cell subgroups (e.g., classifying the cytotoxic natural killer cells expressing CD16) via second antigens including CD16, and generating the actual percentages thereof in the lymphocytes to analyze the immune efficacy and provide a more comprehensive view of all aspects of the immune system.

III. Method of Treating Cancer by Adjusting Content of Lymphocytes

1. Detection of Actual Value of Each of Immune Analysis Indexes

The actual values (or actual numbers) of the immune analysis indexes of the T cells and the natural killer cells were detected through the method of the aforementioned I.1, and the actual value of the natural killer cell cytotoxicity was detected through the method of the aforementioned I.2.

2. Regulation of Actual Value of Immune Analysis Index of Individual to Theoretical Range

2.1. Regulation of Actual Values of Immune Analysis Indexes of T Cells to Theoretical Ranges

A deviation value between an actual value of each of the immune analysis Indexes obtained by the abovementioned III.1 and a corresponding theoretical range was generated according to the abovementioned Table 2. Then, an appropriate dose of the T cells or the specific T cell subgroup to be added to or removed from the individual was then generated according to the blood volume of the individual or other relevant immune analysis indexes.

Specifically, the individual, a cancer patient weighing 65 kg and the estimated total blood volume is 5 L, was taken for an example:

When the actual number of the total number of T cells was 500 cells/μL (the theoretical range of the total number of T cells was 700 cells/μL to 2500 cells/μL), the appropriate dose range of T cells to be added was 1×10⁹ to 1×10¹⁰ [(700-500) cells/μL)×5L=1×10⁹; (2500-500) cells/μL)×5L=1×10¹⁰]. By adjusting the actual total number of T cells to the theoretical range, the individual's immunity was enhanced, thereby improving the safety and the efficacy of the subsequent immune cell therapy.

When the number of CD4-positive T cells was 200 cells/μL, and the number of CD8-positive T cells was 250 cells/μL, the actual value of CD4/CD8 ratio was 0.8:1 (the theoretical range of CD4/CD8 ratio was from 1:1 to 5:1). Since the CD4-positive T cells could activate naive cytotoxic T cells, the ratio of CD4-positive T cells was too low, which resulted in a state of low immunity of the individual. The efficiency of anti-cancer was low when the individual was treated with an immune cell therapy (such as dendritic cell therapy or chimeric antigen receptor T cell therapy). In order to improve the immune efficacy, CD4 positive T cells of the individual were positively screened, and then expanded and cultured, so that the CD4 positive T cells of the individual were increased by at least 2.5×10⁸ cells [(250-200) cells/μL×5L=2.5×10⁸] to restore the actual CD4/CD8 ratio to at least 1:1. By adjusting the actual value of the CD4/CD8 ratio to the theoretical range, the number of CD4 positive T cells was increased, the naive cytotoxic T cells were activated, and the anti-cancer cytotoxicity of the individual was enhanced.

When the actual percentage of naive helper T cells was 25% (the theoretical range of naive helper T cells was higher than 35% (≥35%), and the theoretical range of the total number of T cells was from 700 cells/μL to 2500 cells/μL), a state of low immunity of the individual was presented. In order to improve immunity efficiency, it was necessary to expand the naive helper T cells isolated from the individual to 3.5×10⁸ cells to 1.25×10⁹ cells [(35−25)%×5 L×700 cells/μL=3.5×108; (35−25)%×5L×2500 cells/μL=1.25×10⁹], and then reinfuse the naive helper T cells into the individual, so that the actual percentage of naive helper T cells could be restored to at least 35%. By adjusting the actual percentage of naive helper T cells to the theoretical range, the individual's immune and anti-cancer mechanism could be restarted, the individual's anti-cancer cytotoxicity ability could be improved, and the success rate of the subsequent immune cell therapy could be improved.

When the actual percentage of regulatory helper T cells was 15% (the theoretical range of regulatory helper T cells was from 3% to 10%), the actual number of the total number was 2000 cells/μL, indicating that the regulatory helper T cells used to suppress other lymphocytes were excessive, and the inhibition of the individual's immune and anti-cancer efficacy was resulted. It was necessary to remove 5×10⁸ regulatory helper T cells from the individual [(15−10)%×5L×2000 cells/μL=5×10⁸ cells] to improve the state of excessive suppression of immune efficacy. Specifically, peripheral blood mononuclear cells (PBMC) was extracted from the individual through Hemapheresis, and then at least 3.33×10⁹ cells (5×10⁸ cells, 15%) CD4-positive T cells were positively screened. Furthermore, after negative screening to remove CD25-positive regulatory helper T cells, the remained PBMCs were reinfused to the individual.

Conversely, when the actual percentage of regulatory helper T cells was 2% (the theoretical range of regulatory helper T cells was from 3% to 10%), the actual number of the total number of T cells was 2000 cells/μL, indicating that the regulatory helper T cells used to inhibit other lymphocytes were too less, which could lead to a state of excessive autoimmunity. At this time, the regulatory helper T cells from 1×10⁸ cells/μL to 8×10⁸ cells/μL were necessary to be added to the individual [(3−2)%×5L×2000 cells/μL=1×10⁸; (10−2)%×5 L×2000 cells/μL=8×10⁸] to improve the state of autoimmunity and reduce the dependence on immunosuppressive drugs. Specifically, PBMC could be extracted from the individual through a blood separator, and then the regulatory helper T cells could be positively screened through a lymphocyte separation method. Furthermore, the regulatory helper T cells could be expanded, cultured to 1×10⁸ cells/μL to 8×10⁸ cells/μL and reinfused to the individual.

When the actual percentage of naive cytotoxic T cells was 15% (the theoretical range of naive cytotoxic T cells was >20%), and the actual number of the total number of T cells was 2000 cells/μL, it was indicated that the cytotoxicity of the immune system against the cancer cells was not enough. It was necessary to expand the naive cytotoxic T cells isolated from the individual to at least 5×10⁸ cells [(20−15)%×5 L×2000 cells/μL=5×10⁸ cells], and then reinfuse the naive cytotoxic T cells into the individual, so that the individual's immune and anti-cancer mechanism could be restarted, the individual's anti-cancer cytotoxicity could be improved, and the success rate of the subsequent immune cell therapy could be improved.

2.2. Regulation of Actual Values of Immune Analysis Indexes of Nature Killer Cells to Theoretical Ranges

A deviation value between an actual value of each of the immune analysis Indexes obtained by the abovementioned III.1 and a corresponding theoretical range was generated according to the abovementioned Table 3. Then, an appropriate dose of the nature killer cells or the specific nature killer cell subgroup to be added or removed was then generated according to the blood volume of the individual.

First, the actual value of the natural killer cell cytotoxicity obtained by the abovementioned III.1 and the theoretical range of the aforementioned table 4 were compared, and the trend of the actual values of the natural killer cell cytotoxicity at each of the quantitative ratios and when IL-2 was added and the deviation value of the actual value and the theoretical range were determined. If the actual value of the natural killer cell cytotoxicity did not fall within the theoretical range, a deviation value between an actual value of each of the immune analysis indexes obtained by the abovementioned III.1 and a theoretical range corresponding to the abovementioned Table 3 was generated. Then, an appropriate dose of the nature killer cells or the specific nature killer cell subgroup to be added to or removed from the individual was generated according to the blood volume of the individual, so as to adjust the natural killer cell cytotoxicity to the most optimum condition, and the success rate of the subsequent immune cell therapy could be improved.

Theoretically, when the ratio of E:T increases from 6.25 to 50, the number of cancer cells killed by the natural killer cells were increased in an arithmetic progression, which indicated that the natural killer cells of the individual could kill the cancer cells as usual. In addition, a control group that the natural killer cells were stimulated through IL-2 to perform a maximum activity was used to compare with the group of E:T ratio of 50 to determine whether the individual was in a state of immunosuppression.

Specifically, the individual, a cancer patient weighing 65 kg and the estimated total blood volume is 5 L, was taken for an example, and the following three possible situations were provided as illustrations:
1. If the E:T ratio increased, but the actual value of the cytotoxicity of the natural killer cells was lower than the theoretical range, no upward trend in the arithmetic progress was presented. At the same time, when the actual value of the cytotoxicity of the natural killer cells of the natural killer cells group with the maximum activity group (IL-2 control group) was lower than the theoretical range. The actual percentage of the regulatory natural killer cells obtained by the abovementioned III.1 and the theoretical range corresponding to the Table 3 were further compared, and the actual percentage of the regulatory natural killer cells was much higher:

In this case, it was usually reflected that the regulatory natural killer cells were excessive, and the anti-cancer cytotoxicity of the cytotoxic natural killer cells was excessively inhibited. At this time, according to the actual percentage of the regulatory natural killer cells and the theoretical range corresponding to the Table 3, the excessively high ratio of the regulatory natural killer cells was removed.

For example, when the actual percentage of the regulatory natural killer cells was 15% (the theoretical range of the regulatory natural killer was lower than 10% (≤10%)) and the actual number of the total number of the natural killer cells was 5×10⁸, it was reflected that the regulatory natural killer cells were excessive. 2.5×10⁷ cells (5% of 5×10⁸ cells) of the regulatory natural killer cells were necessary to be removed from the individual to increase the anti-cancer cytotoxicity of the immune system. Specifically, PBMC could be extracted from the individual through a blood separator. Then, the natural killer cells with CD56 positive and CD3 negative could be positively screened through the lymphocyte separation method, and the cell amount was achieved to be 1.67×10⁸ cells (2.5×10⁷/0.15=1.67×10⁸). Then, the remained nature killer cells (mainly cytotoxic natural killer cells) were reinfused to the individual after removing the regulatory nature killer cells (CD16 negative) through a negative selection.
2. as stated above, if the E:T ratio also increased, but the actual values of the cytotoxicity of the natural killer cells in the four groups were lower than the theoretical range, no upward trend in the arithmetic progress was presented. At the same time, when the actual value of the cytotoxicity of the natural killer cells of the group with the maximum activity group of the natural killer cells (IL-2 control group) was lower than the theoretical range, the actual percentages of the regulatory natural killer cells obtained by the abovementioned III.1 and the theoretical ranges corresponding to the Table 3 were further compared, and the actual percentages of the regulatory natural killer cells were within the theoretical ranges:

In this case, it was usually reflected that the individual was in a severely immunocompromised state, often due to (1) aging (particularly for humans over the age of 65 years) (2) the effects of drugs (such as immunosuppressants or chemotherapy) (3) radiation therapy or previous injury by overdose radiation. Usually, the individual was in the situation that the actual number of the total number of the natural killer cells was too low. The actual number of the total number of natural killer cells was increased to enhance the immunity of the individual by expanding the natural killer cells and then reinfusing the natural killer cells into the individual. In some other examples, when the actual percentages of the regulatory natural killer cells obtained by the abovementioned III.1 and the theoretical ranges corresponding to the Table 3 were compared, the actual number of the total number of natural killer cells and the corresponding theoretical range were compared simultaneously to make sure if the actual number of the total number of natural killer cells was within the theoretical range before performing the reinfusion step of the natural killer cells.

For example, when the actual number of the total number of the natural killer cells was 80 cells/μL (the theoretical range of the total number of the natural killer cells was 100 cells/μL), it was reflected that the cytotoxicity of the natural killer cells was insufficient. In order to enhance the cytotoxicity of the natural killer cells of the individual against the cancer cells, the actual number of the total number of the natural killer cells was required to be expanded and cultured to be at least 1×10⁸ cells [(100-80) cells/μL×5 L=1×10⁸ cells], and then reinfused into the individual.
3. if the E:T ratio also increased, but the actual values of the cytotoxicity of natural killer cells in the four groups were lower than the theoretical range, no upward trend in the arithmetic progress was presented. At the same time, when the actual value of the cytotoxicity of the natural killer cells of the group with the maximum activity group of the natural killer cells (IL-2 control group) was within the theoretical range:

In this case, it was represented that the activity of the natural killer cells could still be stimulated and activated to enhance the anti-cancer cytotoxicity though the activity of the natural killer cells of the individual was worse than that of ordinary people. Therefore, the natural killer cells could be isolated from the individual, activated and then cultured by IL-2. The natural killer cells were reinfused into the individual after the actual value of the cytotoxicity of natural killer cells was confirmed to be restored to the theoretical range.

Therefore, it was allowed to provide a more comprehensive analysis of the status of the anti-cancer and anti-virus abilities of the natural killer cells by analyzing the actual value of the cytotoxicity of the natural killer cells, the actual number of the total number of natural killer cells, and the actual percentage of regulatory natural killer cells and the corresponding theoretical ranges. In addition, according to the analysis results, the content of the natural killer cells in the individual or the regulatory natural killer cells in the individual could be adjusted, or the natural killer cells activated by IL-2 could be added, so that the cytotoxicity of the natural killer cells could be restored to the theoretical range, and the success rate of the subsequent other immune cell therapy (such as natural killer cell therapy) was improved.

Although the disclosure has been disclosed in the above embodiments, it is not intended to limit the disclosure, and it is to be understood that those skilled in the art can make various changes and modifications without departing from the spirit and scope of the disclosure. The scope of protection of the present disclosure is subject to the definition of the scope of claims.

What is claimed is:

1. A method for detecting immune efficacy, comprising:
providing an in vitro sample of an individual, wherein the in vitro sample comprises a plurality of lymphocytes;
classifying the plurality of lymphocytes into a plurality of T cell subgroups based on expression or not of a plurality of first antigens, and classifying the plurality of lymphocytes into a plurality of natural killer cell subgroups based on expression or not of a plurality of second antigens, wherein the plurality of first antigens comprise CD28, and the plurality of second antigens comprise CD16;
generating quantity percentages of the plurality of T cell subgroups in the plurality of lymphocytes and quantity percentages of the plurality of nature killer cell subgroups in the plurality of lymphocytes to obtain actual percentages of the plurality of T cell subgroups and actual percentages of the plurality of nature killer cell subgroups; and
judging whether the actual percentage of each of the plurality of T cell subgroups meets a T cell subgroup reference range, and judging whether the actual percentage of each of the plurality of natural killer cell subgroups meets a natural killer cell subgroup reference range,
when the actual percentage of each of the plurality of T cell subgroups meets the T cell subgroup reference range and the actual percentage of each of the plurality of natural killer cell subgroups meets the natural killer cell subgroup reference range, the immune efficacy of the individual is judged normal.

2. The method of claim 1, wherein the plurality of T cell subgroups comprise: an naive helper T cell, an aging helper T cell, a regulatory helper T cell, an naive cytotoxic T cell, an aging cytotoxic T cell, or a combination thereof, wherein
the naive helper T cell is CD3+, CD4+, CD45RA+, CD62L+, and CD28+;
the aging helper T cell is CD3+, CD4+, CD45RO+, and CD62L+;
the regulatory helper T cell is CD3+, CD4+, CD25+, FoxP3+, and CD39+;
the naive cytotoxic T cell is CD8+, CD27+, CD45RA+, CD62L+, and CD127+; and
the aging cytotoxic T cell is CD8+, CD27+, CD45RO+, CD62L+, and CD57+.

3. The method of claim 1, wherein the T cell subgroup reference range comprises a naive helper T cell reference range of from 20% to 100%.

4. The method of claim 1, wherein the T cell subgroup reference range comprises a regulatory helper T cell reference range of from 1% to 15%.

5. The method of claim 1, wherein the T cell subgroup reference range comprises a naive cytotoxic T cell reference range of from 15% to 100%.

6. The method of claim 1, wherein the T cell subgroup reference range comprises an aging cytotoxic T cell reference range of from 0% to 50%.

7. The method of claim 1, wherein the T cell subgroup reference range is derived from physiological values of a healthy population, wherein the healthy population is human individuals with ages from 5 to 85 and does not suffer from immunodeficiency syndrome and hyperimmune syndrome.

8. The method of claim 2, further comprising:
generating a quantity percentage of CD28 positive in the naive helper T cell, the aging helper T cell and the regulatory helper T cell relative to a sum of quantity percentages of the naive helper T cell to obtain an actual percentage of CD28 positive in helper T cells, based on a total weight (100% by weight) of the aging helper T cell and the regulatory helper T cell; and
judging whether the actual percentage of CD28 positive in the helper T cells meets a CD28 positive reference range in the helper T cells.

9. The method of claim 8, wherein the CD28 positive reference range in the helper T cells is from 80% to 100%.

10. The method of claim 8, wherein the CD28 positive reference range in the helper T cells is derived from physiological values of a healthy population, wherein the healthy population is human individuals with ages from 5 to 85 and does not suffer from immunodeficiency syndrome and hyperimmune syndrome.

11. The method of claim 1, wherein the plurality of natural killer cell subgroups comprise a cytotoxic natural killer cell and a regulatory natural killer cell, wherein the cytotoxic natural killer cell is CD16+, CD34+, CD56+, CD94+, and CD117+, and the regulatory natural killer cell is CD34+, CD56+, CD94+, and CD117+.

12. The method of claim 11, wherein the natural killer cell subgroup reference range comprises a regulatory natural killer cell reference range of from 0% to 20%.

13. The method of claim 12, wherein the regulatory natural killer cell reference range is derived from physiological values of a healthy population, wherein the healthy population is human individuals with ages from 5 to 85 and does not suffer from immunodeficiency syndrome and hyperimmune syndrome.

14. The method of claim 1, further comprising:
determining an actual value of a natural killer cell cytotoxicity of the plurality of natural killer cells, wherein the actual value of the natural killer cell cytotoxicity is calculated by following steps:
co-culturing the plurality of natural killer cells with a plurality of cancer cells with a quantitative ratio; and
generating a quantity percentage of the cancer cells that die to obtain the actual value of the natural killer cell cytotoxicity; and
judging whether the actual value of the natural killer cell cytotoxicity meets a natural killer cell cytotoxicity reference range, wherein the natural killer cell cytotoxicity reference range is from 0% to 58.8% when the quantitative ratio is 6.25, the natural killer cell cytotoxicity reference range is from 1.7% to 88.7% when the quantitative ratio is 12.5, the natural killer cell cytotoxicity reference range is from 17.4% to 100% when the quantitative ratio is 25, and the natural killer cell cytotoxicity reference range is from 35.3% to 100% when the quantitative ratio is 50.

15. The method of claim 1, further comprising:
cryopreserving each of the plurality of T cell subgroups when the actual percentage of each of the plurality of T cell subgroups meets the T cell subgroup reference range; and
cryopreserving each of the plurality of natural killer cell subgroups when the actual percentage of each of the plurality of natural killer cell subgroups meets the natural killer cell subgroup reference range.

16. The method of claim 15, wherein cryopreserving each of the plurality of T cell subgroups or cryopreserving each of the plurality of natural killer cell subgroups comprises:
sorting each of the plurality of T cell subgroups or each of the plurality of natural killer cell subgroups by an immunomagnetic bead cell sorting method; and
cryopreserving each of the plurality of T cell subgroups or each of the plurality of natural killer cell subgroups.

17. A system for detecting immune efficacy, comprising a processor and a memory, and the memory storing a plurality of computer program instructions, and the computer program instructions, when executed by the processor, causing the processor to implement following steps:
accessing an in vitro sample data of an individual, the in vitro sample data comprising a plurality of sample cell surface antigen data;
generating a plurality of T cell subgroup count data and a plurality of natural killer cell subgroup count data according to the in vitro sample data using a cell subgroup database, wherein the cell subgroup database comprises a T cell subgroup classification information and a natural killer cell subgroup classification information, and the T cell subgroup classification information comprises a classification index according to expression or not of a plurality of first antigens, wherein the plurality of first antigens comprise CD28, and the natural killer cell subgroup classification information comprises a classification index according to expression or not of a plurality of second antigens, wherein the plurality of second antigens comprise CD16; and
generating an immune efficacy data according to the plurality of T cell subgroup count data and the plurality of natural killer cell subgroup count data using an immune efficacy evaluation database, wherein the immune efficacy evaluation database comprises a plurality of T cell subgroup reference range data and a plurality of natural killer cell subgroup reference range data.

18. The system of claim 17, wherein the plurality of first antigens further comprise CD3, CD4, CD8, CD25, CD27, CD39, CD45RO, CD45RA, CD57, CD62L, CD127, FoxP3, or a combination thereof, and the plurality of second antigens further comprise CD34, CD56, CD94, CD117, or a combination thereof.

19. The system of claim 17, further comprising an output module connected to the processor, and the output module receiving the immune efficacy data and outputs an immune efficacy report.

20. The system of claim 19, wherein the immune efficacy report comprises an immune efficacy field and a plurality of immune analysis index fields.

21. A method of treating a cancer of an individual by a plurality of lymphocytes, wherein the plurality of lymphocytes comprise a plurality of T cells, a plurality of natural killer cells or a combination thereof, and the method comprises:
(a) generating a deviation value of a total number of T cells between an actual number of the total number of T cells and a theoretical range of the total number of T cells, and generating a deviation value of a total number of nature killer cells between an actual number of the total number of nature killer cells and a theoretical range of the total number of nature killer cells; and
(b) administering a pharmaceutical composition comprising the plurality of T cells to the individual based on the deviation value of the total number of T cells to allow the actual number of the total number of T cells of the individual to fall within the theoretical range of the total number of T cells, and
administering the pharmaceutical composition comprising the plurality of nature killer cells to the individual based on the deviation value of the total number of nature killer cells to allow the actual number of the total number of nature killer cells of the individual to fall within the theoretical range of the total number of nature killer cells.

22. The method of claim 21, wherein the theoretical range of the total number of T cells is from 700 cells/μL to 2500 cells/μL, and the theoretical range of the total number of nature killer cells is from 100 cells/μL to 300 cells/μL.

23. The method of claim 21, further comprises:
(a) classifying the plurality of lymphocytes into a plurality of T cell subgroups based on expression or not of a plurality of first antigens, and classifying the plurality of lymphocytes into a plurality of natural killer cell subgroups based on expression or not of a plurality of second antigens;
(b) generating a deviation value of a T cell subgroup between an actual percentage of the T cell subgroup and a theoretical range of the T cell subgroup, and generating a deviation value of a nature killer cell subgroup between an actual percentage of the nature killer cell subgroup and a theoretical range of the nature killer cell subgroup, wherein the actual percentage of the T cell subgroup is an actual quantity percentage of each of the plurality of T cell subgroups in the plurality of lymphocytes, and the actual percentage of the nature killer cell subgroup is an actual quantity percentage of each of the plurality of nature killer cell subgroups in the plurality of lymphocytes; and
(c) administering the pharmaceutical composition comprising the plurality of T cell subgroups to the individual based on the deviation value of the T cell subgroup to allow the actual percentage of the T cell subgroup of the individual to fall within the theoretical range of the T cell subgroup, and
administering the pharmaceutical composition comprising the plurality of nature killer cells to the individual based on the deviation value of the nature killer cell subgroup to allow the actual percentage of the nature killer cell subgroup of the individual to fall within the theoretical range of the nature killer cell subgroup.

24. The method of claim 23, wherein the plurality of T cell subgroups comprise: a naive helper T cell, a regulatory helper T cell, a naive cytotoxic T cell, or a combination thereof, wherein
the naive helper T cell is CD3+, CD4+, CD45RA+, CD62L+, and CD28+;
the regulatory helper T cell is CD3+, CD4+, CD25+, FoxP3+, and CD39+; and
the naive cytotoxic T cell is CD8+, CD27+, CD45RA+, CD62L+, and CD127+.

25. The method of claim 24, wherein the theoretical range of the T cell subgroup comprises a theoretical range of the naive helper T cell, a theoretical range of the regulatory helper T cell, a theoretical range of the naive cytotoxic T cell, or a combination thereof, wherein the theoretical range of the naive helper T cell is from 35% to 100%, the theoretical range of the regulatory helper T cell is from 3% to 10%, and the theoretical range of the naive cytotoxic T cell is from 20% to 100%.

26. The method of claim 23, wherein the plurality of natural killer cell subgroups comprise a regulatory natural killer cell, wherein the regulatory natural killer cell is CD34+, CD56+, CD94+, and CD117+.

27. The method of claim 26, wherein the theoretical range of the nature killer cell subgroup comprises a theoretical range of the regulatory natural killer cell, wherein the theoretical range of the regulatory natural killer cell is from 0% to 10%.

28. The method of claim 21, further comprises:
(a) classifying the plurality of lymphocytes into a plurality of T cell subgroups based on expression or not of a plurality of first antigens, wherein the plurality of T cell subgroups comprise a first T cell subgroup and a second T cell subgroup;
(b) generating a deviation value of a ratio of T cell subgroups between an actual value of a ratio of two kinds of T cell subgroups and a theoretical range of the ratio of two kinds of T cell subgroups, wherein the actual value of the ratio of two kinds of T cell subgroups is a quantitative ratio of the first T cell subgroup and the second T cell subgroup; and
(c) administering the pharmaceutical composition comprising the first T cell subgroup or the second T cell subgroup to the individual based on the deviation value of the ratio of the T cell subgroups to allow the actual value of the ratio of two kinds of T cell subgroups of the individual to fall within the theoretical range of the ratio of two kinds of T cell subgroups.

29. The method of claim 28, wherein the theoretical range of the ratio of two kinds of T cell subgroups is from 1:1 to 5:1 when the first T cell subgroup is CD4 positive T cells and the second T cell subgroup is CD8 positive T cells.

30. The method of claim 21, further comprises performing an immune cell therapy on the individual after the step of administering the pharmaceutical composition comprising the plurality of T cells to the individual or administering the pharmaceutical composition comprising the plurality of nature killer cells to the individual.

31. The method of claim 30, wherein the immune cell therapy comprises a natural killer cell therapy, a cytokine-induced killer cell therapy, a γδ T cell therapy, a dendritic cell therapy, a tumor infiltrating lymphocyte therapy, a chimeric antigen receptor T cell therapy, or a combination thereof.

32. The method of claim 21, wherein the plurality of lymphocytes are derived from the individual and obtained by culturing in vitro.

33. The method of claim 21, wherein the cancer comprises colorectal cancer, ovarian cancer, prostate cancer, breast cancer, brain cancer, cervical cancer, bladder cancer, anal cancer, uterine cancer, colon cancer, liver cancer, pancreatic cancer, lung cancer, endometrial cancer, bone cancer, testicular cancer, skin cancer, kidney cancer, stomach cancer, esophagus cancer, head and neck cancer, salivary gland cancer, hepatocellular carcinoma, non-small cell lung cancer, head and neck squamous cell cancer, basal cell cancer, cutaneous squamous cell cancer, cholangiocarcinoma, merkel cell carcinoma or a combination thereof.

34. A method of treating a cancer of an individual by a plurality of nature killer cells, comprises:
(a) determining an actual value of a natural killer cell cytotoxicity of the plurality of natural killer cells, wherein the actual value of the natural killer cell cytotoxicity is generated by following steps:
co-culturing the plurality of natural killer cells with a plurality of cancer cells with a quantitative ratio; and
generating a quantity percentage of the cancer cells that die to obtain the actual value of the natural killer cell cytotoxicity;
(b) generating a deviation value of a natural killer cell cytotoxicity between an actual value of the natural killer cell cytotoxicity and a theoretical range of the natural killer cell cytotoxicity based on the actual value of the natural killer cell cytotoxicity and the theoretical range of the natural killer cell cytotoxicity;
(c) administering a pharmaceutical composition comprising the plurality of nature killer cells to the individual based on the deviation value of the natural killer cell cytotoxicity to allow the actual value of the natural killer cell cytotoxicity of the individual to fall within the theoretical range of the natural killer cell cytotoxicity.

35. The method of claim 34, wherein the theoretical range of the natural killer cell cytotoxicity is from 0% to 58.8% when the quantitative ratio is 6.25, the theoretical range of the natural killer cell cytotoxicity is from 1.7% to 88.7% when the quantitative ratio is 12.5, the theoretical range of the natural killer cell cytotoxicity is from 17.4% to 100% when the quantitative ratio is 25, and the theoretical range of the natural killer cell cytotoxicity is from 35.3% to 100% when the quantitative ratio is 50.

36. The method of claim 35, wherein at the step of (a), the theoretical range of the natural killer cell cytotoxicity is 57.4% to 100% when the quantitative ratio is 50, and interleukin-2 with an action concentration of 100 international units/mL is added to co-culture with the plurality of natural killer cells and the cancer cells.

37. The method of claim 35, further comprises:
generating a deviation value of a total number of nature killer cells between an actual number of the total number of nature killer cells and a theoretical range of the total number of nature killer cells; and
administering the pharmaceutical composition comprising the plurality of nature killer cells to the individual based on the deviation value of the natural killer cell cytotoxicity and the deviation value of the total number of nature killer cells to allow the actual number of the total number of nature killer cells of the individual to fall within the theoretical range of the total number of nature killer cells, thereby allowing the actual value of the natural killer cell cytotoxicity to fall within the theoretical range of the natural killer cell cytotoxicity.

38. The method of claim 35, further comprises:
classifying the plurality of nature killer cells into a plurality of natural killer cell subgroups based on expression or not of a plurality of antigens, wherein the plurality of natural killer cell subgroups comprise a regulatory natural killer cell, wherein the regulatory natural killer cell is CD34+, CD56+, CD94+, and CD117+;
generating a deviation value of a regulatory natural killer cell between an actual percentage of the regulatory natural killer cell and a theoretical range of the regulatory natural killer cell, wherein the actual percentage of the regulatory natural killer cell is an actual quantity percentage of the regulatory natural killer cell in lymphocytes;
administering the pharmaceutical composition comprising the regulatory natural killer cell to the individual based on the deviation value of the natural killer cell cytotoxicity and the deviation value of the regulatory natural killer cell to allow the actual percentage of the regulatory natural killer cell of the individual to fall within the theoretical range of the regulatory natural killer cell, thereby allowing the actual value of the natural killer cell cytotoxicity to fall within the theoretical range of the natural killer cell cytotoxicity.
